(19) United States
(12) Patent Application Publication
(10) Pub. No.: US 2026/0196360 A1
(43) Pub. Date: Jul. 9, 2026

(54) MEDICAL SUPPORT DEVICE, MEDICAL SUPPORT METHOD, AND COMPUTER-READABLE RECORDING MEDIUM

(71) Applicant: OLYMPUS MEDICAL SYSTEMS CORP., Tokyo (JP)

(72) Inventors: Masakazu WATANABE, Tokyo (JP); Keiji OKUMURA, Tokyo (JP); Yukihito FURUHASHI, Tokyo (JP); Takashi SUGIMOTO, Tokyo (JP)

(73) Assignee: OLYMPUS MEDICAL SYSTEMS CORP., Tokyo (JP)

(21) Appl. No.: 19/554,864

(22) Filed: Mar. 3, 2026

Related U.S. Application Data
(63) Continuation of application No. PCT/JP2023/032757, filed on Sep. 7, 2023.

(51) Int. Cl. G16H 70/20; A61B 1/00; G16H 50/70

(52) U.S. Cl. CPC G16H 70/20; G16H 50/70; A61B 1/00059

(57) ABSTRACT

A medical support device includes a processor including hardware. The processor is configured to: perform division on endoscope-related procedure information with a division method according to content of the endoscope-related procedure information to extract a specific word; compare the specific word with a standard term; determine the specific word as an attribute value when the specific word matches the standard term;
determine and accumulate the specific word as a facility-specific term when the specific word does not match the standard term; and update, as the standard term, the accumulated facility-specific term.

### CROSS REFERENCES TO RELATED APPLICATIONS

This application is a continuation of International Application No. PCT/JP2023/032757, filed on Sep. 7, 2023, the entire contents of which are incorporated herein by reference.

### BACKGROUND

### 1. Technical Field

The present disclosure relates to a medical support device, a medical support method, and a computer-readable recording medium that analyze endoscope-related procedure information.

### 2. Related Art

In the related art, a technique of, after an endoscope examination using an endoscope is performed, sequentially recording and managing a medical data file including an examination report in which an endoscope image and an opinion of a doctor are associated with patient data is known (refer to, for example, Japanese Patent No. 4350294). This technique allows a doctor to search for a past examination of the same patient, refer to a past opinion, and view an image. Thereby, diagnosis and treatment of the patient can be efficiently performed.

### SUMMARY

In some embodiments, a medical support device includes a processor including hardware. The processor is configured to: perform division on endoscope-related procedure information with a division method according to content of the endoscope-related procedure information to extract a specific word; compare the specific word with a standard term; determine the specific word as an attribute value when the specific word matches the standard term; determine and accumulate the specific word as a facility-specific term when the specific word does not match the standard term; and update, as the standard term, the accumulated facility-specific term.

In some embodiments, provided is a medical support method including: performing division on endoscope-related procedure information with a division method according to content of the endoscope-related procedure information to extract a specific word; comparing the specific word with a standard term; determining the specific word as an attribute value when the specific word matches the standard term; determining and accumulating the specific word as a facility-specific term when the specific word does not match the standard term; and updating, as the standard term, the accumulated facility-specific term.

In some embodiments, provided is a non-transitory computer-readable recording medium with an executable program stored thereon. The program causes a processor of a medical support device to execute: performing division on endoscope-related procedure information with a division method according to content of the endoscope-related procedure information to extract a specific word; comparing the specific word with a standard term; determining the specific word as an attribute value when the specific word matches the standard term; determining and accumulating the specific word as a facility-specific term when the specific word does not match the standard term; and updating, as the standard term, the accumulated facility-specific term.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an overall configuration of a medical support system according to a first embodiment of the present disclosure;

FIG. 2 is a block diagram illustrating a functional configuration of a medical support device according to the first embodiment of the present disclosure;

FIG. 3 is a diagram illustrating an example of endoscope-related procedure information recorded in an endoscope-related procedure information DB according to the first embodiment of the present disclosure;

FIG. 4 is a diagram illustrating an example of system setting information recorded in a system setting DB according to the first embodiment of the present disclosure;

FIG. 5 is a diagram illustrating an example of a standard term (qualitative diagnosis) according to the first embodiment of the present disclosure;

FIG. 6 is a diagram illustrating an example of a standard term (treatment) according to the first embodiment of the present disclosure;

FIG. 7 is a diagram illustrating an example of a database in a JED form in the endoscope-related procedure information according to the first embodiment of the present disclosure;

FIG. 8 is a flowchart illustrating an outline of processing executed by the medical support device according to the first embodiment of the present disclosure;

FIG. 9 is a sub-flowchart illustrating an outline of standard term extraction processing in step S106 of FIG. 8;

FIG. 10 is a diagram schematically illustrating an example of standard term extraction processing by a standard term extraction unit included in the medical support device according to the first embodiment of the present disclosure;

FIG. 11 is a diagram schematically illustrating another example of standard term extraction processing by a standard term extraction unit included in the medical support device according to the first embodiment of the present disclosure;

FIG. 12 is a diagram schematically illustrating another example of standard term extraction processing by a standard term extraction unit included in the medical support device according to the first embodiment of the present disclosure;

FIG. 13 is a diagram schematically illustrating another example of standard term extraction processing by a standard term extraction unit included in the medical support device according to the first embodiment of the present disclosure;

FIG. 14 is a diagram illustrating an example of an analysis result analyzed by an analysis/interpretation unit included in the medical support device according to the first embodiment of the present disclosure;

FIG. 15 is a diagram illustrating another example of an interpretation result interpreted by the analysis/interpretation unit included in the medical support device according to the first embodiment of the present disclosure;

FIG. 16 is a diagram illustrating another example of an interpretation result interpreted by an analysis/interpretation unit included in the medical support device according to the first embodiment of the present disclosure;

FIG. 17 is a diagram illustrating another example of an interpretation result interpreted by an analysis/interpretation unit included in the medical support device according to the first embodiment of the present disclosure;

FIG. 18 is a diagram illustrating another example of an interpretation result interpreted by an analysis/interpretation unit included in the medical support device according to the first embodiment of the present disclosure;

FIG. 19 is a block diagram illustrating a functional configuration of a medical support device according to a second embodiment of the present disclosure;

FIG. 20 is a diagram illustrating an example of system setting information recorded in a system setting DB of the medical support device according to the second embodiment of the present disclosure;

FIG. 21 is a flowchart illustrating an outline of processing executed by the medical support device according to the second embodiment of the present disclosure;

FIG. 22 is a sub-flowchart illustrating an outline of standard term extraction processing in step S306 of FIG. 21;

FIG. 23 is a diagram schematically illustrating an example of standard term extraction processing by a standard term extraction unit included in the medical support device according to the second embodiment of the present disclosure;

FIG. 24 is a diagram schematically illustrating another example of standard term extraction processing by a standard term extraction unit included in the medical support device according to the second embodiment of the present disclosure;

FIG. 25 is a diagram schematically illustrating another example of standard term extraction processing by a standard term extraction unit included in the medical support device according to the second embodiment of the present disclosure;

FIG. 26 is a diagram schematically illustrating another example of standard term extraction processing by a standard term extraction unit included in the medical support device according to the second embodiment of the present disclosure;

FIG. 27 is a sub-flowchart illustrating an outline of the facility-specific term extraction processing in step S307 of FIG. 21;

FIG. 28 is a diagram schematically illustrating an example of facility-specific term extraction processing by a facility-specific term extraction unit included in the medical support device according to the second embodiment of the present disclosure;

FIG. 29 is a diagram illustrating an example of data which is input in a case where an attribute value of acquired data is accumulated in an attribute value accumulation DB included in the medical support device according to the second embodiment of the present disclosure;

FIG. 30 is a diagram illustrating an example of standard terms (qualitative diagnosis) accumulated in a standard term accumulation DB included in the medical support device according to the second embodiment of the present disclosure;

FIG. 31 is a diagram illustrating an example of standard terms (treatment) accumulated in the standard term accumulation DB included in the medical support device according to the second embodiment of the present disclosure;

FIG. 32 is a diagram illustrating an example of qualitative diagnosis and treatment of attribute values accumulated in the attribute value accumulation DB included in the medical support device according to the second embodiment of the present disclosure;

FIG. 33 is a block diagram illustrating a functional configuration of a medical support device according to a third embodiment of the present disclosure;

FIG. 34 is a diagram illustrating an example of system setting information recorded in a system setting DB 141B included in the medical support device according to the third embodiment of the present disclosure;

FIG. 35 is a flowchart illustrating an outline of processing executed by a medical support device 10B included in the medical support device according to the third embodiment of the present disclosure;

FIG. 36 is a block diagram illustrating a functional configuration of a medical support device according to a fourth embodiment of the present disclosure;

FIG. 37 is a diagram illustrating an example of standard term (treatment) use information of the medical support device according to the fourth embodiment of the present disclosure;

FIG. 38 is a diagram illustrating an example of facility setting information of the medical support device according to the fourth embodiment of the present disclosure;

FIG. 39 is a flowchart illustrating an outline of processing executed by the medical support device according to the fourth embodiment of the present disclosure;

FIG. 40 is a diagram schematically illustrating a situation where the number of uses is counted up in a case where a standard term extraction unit included in the medical support device according to the fourth embodiment of the present disclosure acquires an attribute extracted in the standard term extraction processing;

FIG. 41 is a sub-flowchart illustrating an outline of the facility-specific term extraction processing in step S710 of FIG. 39;

FIG. 42 is a diagram schematically illustrating an example of facility-specific term extraction processing by a facility-specific term extraction unit included in the medical support device according to the fourth embodiment of the present disclosure;

FIG. 43 is a diagram schematically illustrating standard term adjustment by a standard term adjustment unit included in the medical support device according to the fourth embodiment of the present disclosure;

FIG. 44 is a block diagram illustrating a functional configuration of a medical support device according to a fifth embodiment of the present disclosure;

FIG. 45 is a flowchart illustrating an outline of standard term extraction processing executed by the medical support device according to the fifth embodiment of the present disclosure;

FIG. 46 is a diagram illustrating an example of a character string which is extracted by a standard term extraction unit included in the medical support device according to the fifth embodiment of the present disclosure and is accumulated in a standard term temporary accumulation DB;

FIG. 47 is a flowchart illustrating an outline of adjustment processing executed by a standard term adjustment unit included in the medical support device according to the fifth embodiment of the present disclosure;

FIG. 48 is a diagram illustrating an example of an analysis/interpretation result analyzed/interpreted by the analysis/interpretation unit included in the medical support device according to the embodiment of the present disclosure;

FIG. 49 is a diagram illustrating another example of an analysis/interpretation result analyzed/interpreted by the analysis/interpretation unit included in the medical support device according to the embodiment of the present disclosure;

FIG. 50 is a diagram illustrating another example of an analysis/interpretation result analyzed/interpreted by the analysis/interpretation unit included in the medical support device according to the embodiment of the present disclosure;

FIG. 51 is a diagram illustrating another example of an analysis/interpretation result analyzed/interpreted by the analysis/interpretation unit included in the medical support device according to the embodiment of the present disclosure;

FIG. 52 is a diagram illustrating another example of an analysis/interpretation result analyzed/interpreted by the analysis/interpretation unit included in the medical support device according to the embodiment of the present disclosure; and

FIG. 53 is a diagram illustrating another example of an analysis/interpretation result analyzed/interpreted by the analysis/interpretation unit included in the medical support device according to the embodiment of the present disclosure.

### DETAILED DESCRIPTION

Hereinafter, modes for carrying out the present disclosure will be described in detail with reference to the drawings. Note that the present disclosure is not limited to the following embodiments. In addition, each drawing referred to in the following description merely schematically illustrates a shape, a size, and a positional relationship to an extent that the content of the present disclosure can be understood. That is, the present disclosure is not limited only to the shape, the size, and the positional relationship illustrated in each drawing. Further, in the description of the drawings, the same portions will be denoted by the same reference numerals.

### First Embodiment

### Overall Configuration of Medical Support System

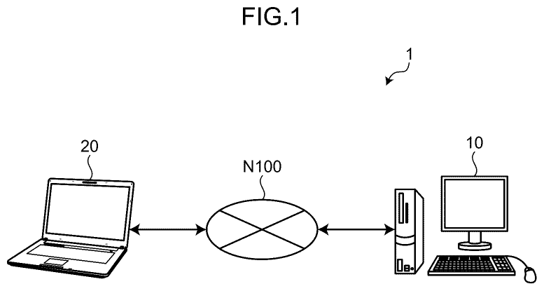

FIG. 1 is a diagram illustrating an overall configuration of a medical support system according to a first embodiment of the present disclosure.

A medical support system 1 illustrated in FIG. 1 includes a medical support device 10, and an endoscope management device 20.

The medical support device 10 acquires, as patient data, endoscope-related procedure information including a plurality of characters and being related to at least one of an endoscope image captured by an endoscope, an opinion of a doctor, a qualitative diagnosis by using an endoscope, or a treatment, from the endoscope management device 20, extracts a specific word, and analyzes the extracted specific word.

The endoscope management device 20 records and manages, as the patient data, endoscope-related procedure information (hereinafter, simply referred to as “endoscope-related procedure information”) including a plurality of characters and being related to one of an endoscope image captured by an endoscope, an opinion of a doctor, a qualitative diagnosis by using an endoscope, and a treatment. Note that, in FIG. 1, although the endoscope management device 20 will be described as one device, the present disclosure is not limited thereto, and a plurality of endoscope management devices may be provided, or one or a plurality of endoscope management devices 20 may be provided for each facility. The endoscope management device 20 is configured using, for example, a facility server or an endoscope server.

The medical support device 10 and the endoscope management device 20 are bidirectionally connected to each other via a network N100. The network N100 is configured with, for example, an Internet network, a mobile network, or the like. In addition, the medical support device 10 may be configured to acquire the endoscope-related procedure information from the endoscope management device 20 not only through the network N100 but also through a storage medium such as a memory card. Further, the medical support device 10 may be configured such that a part of the processing is performed by another server (not illustrated) connected to the network.

### Functional Configuration of Medical Support Device

Next, a functional configuration of the medical support device 10 will be described.

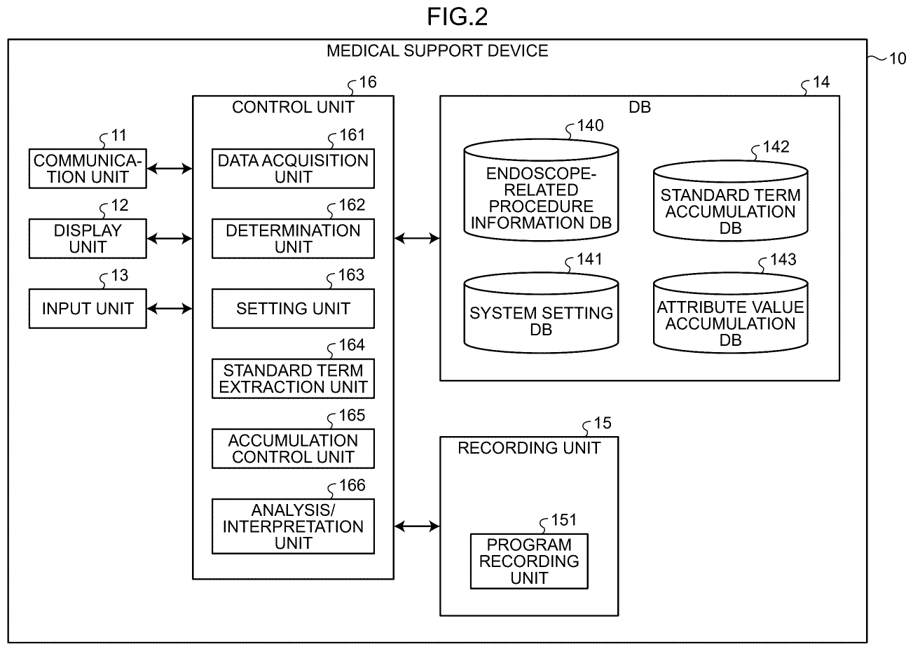

FIG. 2 is a block diagram illustrating a functional configuration of the medical support device 10.

The medical support device 10 illustrated in FIG. 2 includes a communication unit 11, a display unit 12, an input unit 13, a database 14 (hereinafter, simply referred to as “DB 14”), a recording unit 15, and a control unit 16.

Under a control of the control unit 16, the communication unit 11 transmits various types of information to the endoscope management device 20 via the network N100, and receives various types of information from the endoscope management device 20. The communication unit 11 is configured using a communication module.

The display unit 12 displays various types of information related to the medical support device 10 under the control of the control unit 16. The display unit 12 is configured using a liquid crystal display, an organic electroluminescent display (EL display), or the like.

The input unit 13 receives various types of input in response to an operation from the outside, and outputs the input to the control unit 16. The input unit 13 is configured using a button, a keyboard, a switch, a touch panel, or the like.

The DB 14 records various types of information. The DB 14 is configured using a hard disk drive (HDD), a solid state drive (SSD), or the like. The DB 14 includes an endoscope-related procedure information DB 140, a system setting DB 141, a standard term accumulation DB 142, and an attribute value accumulation DB 143.

The endoscope-related procedure information DB 140 records the endoscope-related procedure information which is acquired from the endoscope management device 20 by a data acquisition unit 161 to be described later via the communication unit 11 and the network N100.

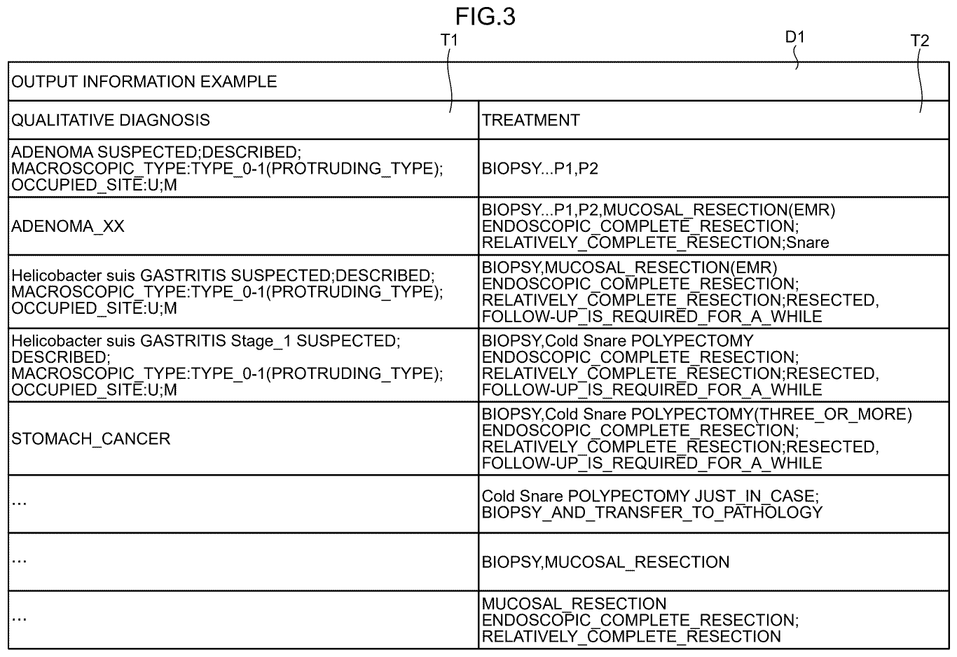

FIG. 3 is a diagram illustrating an example of the endoscope-related procedure information recorded in the endoscope-related procedure information DB 140.

The endoscope-related procedure information D1 illustrated in FIG. 3 is data configured in a predetermined form, and includes data which includes at least a plurality of characters (text, symbol, space, comma, and the like) and is related to a qualitative diagnosis by using an endoscope and a treatment. The endoscope-related procedure information D1 is, for example, data in a Japan endoscopic database (JED) form or data in a CSV form. The endoscope-related procedure information D1 is data formed by a combination of an endoscope examination type defined by the Japan Gastroenterological Endoscopy Society and an output form type. Here, the endoscope examination type includes at least an upper endoscope, a large intestine endoscope, a small intestine endoscope, and a biliary-pancreatic endoscope. In addition, the output form includes at least Type1, Type2, and Type4. Further, the endoscope-related procedure information D1 includes qualitative diagnosis text data T1 and treatment text data T2. For example, “adenoma suspected;described;macroscopic_type:type_0-1(protruding_type);occupied_site:U;M” is stored in a first line of the qualitative diagnosis text data T1. In addition, “biopsy . . . P1,P2” is stored in a first line of the treatment text data T2. Note that patient data and doctor opinion data are omitted from the endoscope-related procedure information D1 of FIG. 3 in order to simplify the description.

The system setting DB 141 records system setting information in which a character division method, an attribute, and additional information extracted from the endoscope-related procedure information are associated. Note that the additional information includes low-level information associated with the attribute, such as an attribute value “1” (first attribute) and an attribute value “2” (second attribute) of the JED form data.

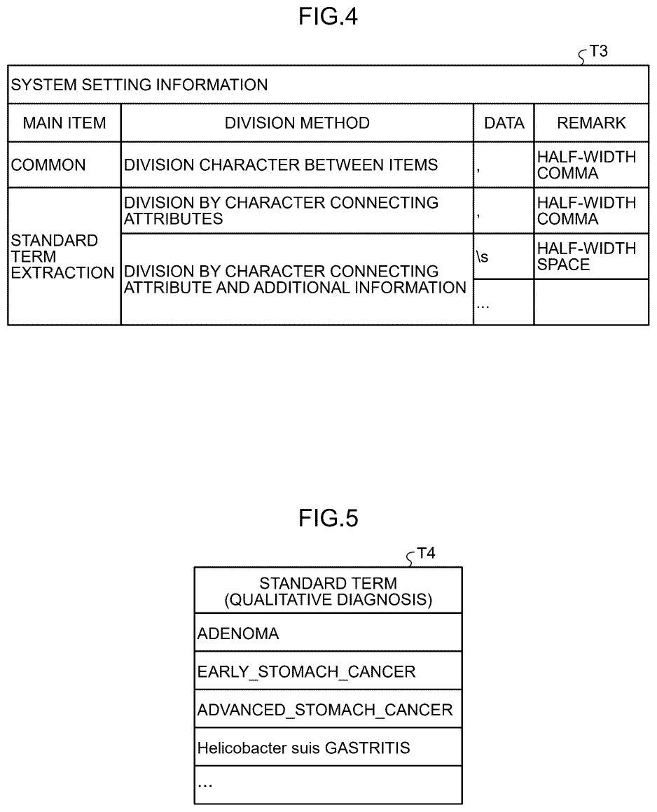

FIG. 4 is a diagram illustrating an example of system setting information recorded in the system setting DB 141.

The system setting information T3 illustrated in FIG. 4 stores, as information for dividing and extracting, a connection character between items, a connection character between attributes, and a connection character between an attribute and additional information in the endoscope-related procedure information. Specifically, the system setting information T3 stores the contents of a division method, data, and remark in association with main items. For example, a division method “division character between items”, data “,”, and remark “half-width comma” are stored in association with a main item “common” of the system setting information T3. In addition, division methods “division by character connecting attributes” and “division by character connecting attribute and additional information”, data “,”, “,”, “\s”, and “ . . . ”, and remark “half-width comma”, “half-width comma”, and “half-width space” are stored in association with a main item “standard term extraction” of the system setting information T3.

The standard term accumulation DB 142 accumulates standard terms for comparison with characters extracted from the endoscope-related procedure information.

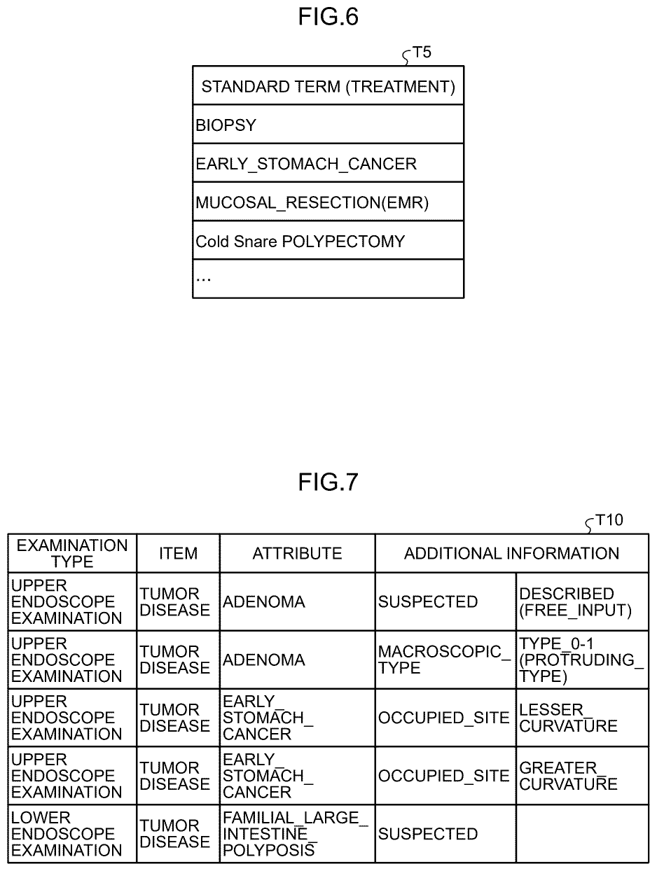

FIG. 5 is a diagram illustrating an example of a standard term (qualitative diagnosis). FIG. 6 is a diagram illustrating an example of a standard term (treatment).

The standard term T4 illustrated in FIG. 5 and the standard term T5 illustrated in FIG. 6 store terms (attributes) defined by the JED form. For example, in a first line of the standard term T4 illustrated in FIG. 5, “adenoma” is stored as the standard term (qualitative diagnosis). Note that, in the standard term T4 illustrated in FIG. 5 and the standard term T5 illustrated in FIG. 6, although the terms (attributes) defined by the JED format are exemplified as an example, the present disclosure is not limited thereto, and terms (attributes) defined in each industry can be appropriately set as the standard terms.

In a case where a standard term extraction unit 164 determines that the specific word (divided characters) matches the standard term, an accumulation control unit 165 to be described later accumulates, as an attribute value, the specific word (divided characters) in the attribute value accumulation DB 143.

The recording unit 15 is configured using a volatile memory, a nonvolatile memory, an HDD, an SSD, or the like. The recording unit 15 includes a program recording unit 151.

The program recording unit 151 records various programs and parameters for operating the medical support device 10.

The control unit 16 corresponds to a processor according to the present disclosure. The control unit 16 is implemented by using a processor that includes hardware such as a field-programmable gate array (FPGA), a graphics processing unit (GPU), or a central processing unit (CPU) and a memory that is a temporary storage area used by the processor. In addition, the control unit 16 comprehensively controls each unit included in the medical support device 10. The control unit 16 includes a data acquisition unit 161, a determination unit 162, a setting unit 163, the standard term extraction unit 164, an accumulation control unit 165, and an analysis/interpretation unit 166.

The data acquisition unit 161 acquires the endoscope-related procedure information that includes a plurality of characters and is related to at least one of qualitative diagnosis by using an endoscope or treatment and has a predetermined form, from the endoscope management device 20 via the communication unit 11.

The determination unit 162 determines whether or not there is acquired data from the data acquisition unit 161. Specifically, the determination unit 162 determines whether or not the acquired data from the data acquisition unit 161 includes one-row acquired data in units of one row and one lesion.

The setting unit 163 acquires system setting information recorded in the system setting DB 141. Specifically, the setting unit 163 acquires, from the system setting DB 141, system setting information T3 (refer to FIG. 4) in which a connection character between items, a connection character between attributes, and a connection character between an attribute and additional information in the endoscope-related procedure information are stored as information for dividing and extracting.

The standard term extraction unit 164 extracts a specific word (divided characters or a character string) by dividing the endoscope-related procedure information by using the system setting information T3. In addition, the standard term extraction unit 164 determines whether or not the specific word (divided characters) matches the standard term. Further, the standard term extraction unit 164 divides the endoscope-related procedure information from a rear side by a character connecting an attribute and additional information, according to the standard term T4 (refer to FIG. 5) or the standard term T5 which is set by the setting unit 163.

The accumulation control unit 165 registers, as an attribute value, the specific word which is determined as matching the standard term by the standard term extraction unit 164, in the attribute value accumulation DB 143.

The analysis/interpretation unit 166 performs analysis or interpretation on the attribute value on the basis of attribute value accumulation data that is accumulated in the attribute value accumulation DB 143, and displays an analysis result or an interpretation result on the display unit 12.

### Details of Database in JED Form

Next, details of the database in the JED form that is the endoscope-related procedure information will be described.

FIG. 7 is a diagram illustrating an example of the database in the JED form that is related to the endoscope-related procedure information.

Here, the JED is a database specialized for endoscopes that is promoted by the Japan Gastroenterological Endoscopy Society. In a format T10 illustrated in FIG. 7 that is the JED form of the endoscope-related procedure information, an item, an attribute, an attribute “1”, and an attribute “2” are stored in association with each other for each examination type. For example, in a first line of the format T10 that is the JED form, an item “tumor disease”, an attribute “tumor”, and additional information “suspected” and “described (free input)” are stored in association with an examination type “upper endoscope examination”.

Meanwhile, there are a plurality of manufacturers with systems capable of outputting data in the JED form, and the output form, the term selection, and the term input are different for each system. For example, a certain manufacturer defines attributes for diagnosing stomach cancer in the system as early stomach cancer and advanced stomach cancer. On the other hand, other manufacturers define an attribute as stomach cancer. For this reason, in the format T10 that is the JED form, there is a difference in terms for each manufacturer and system.

For this reason, in the related art, in a case of a hierarchical structure including items, attributes, and additional information (attribute “1” and attribute “2”), which are appropriately set in the JED form, it is difficult to recognize which word in which location needs to be set as an analysis target (extraction target). That is, in the related art, even in a case where data is output in the format that is the JED form, it is difficult to accurately extract, count, and analyze all input terms due to the following reason 1 and reason 2.

The reason 1 is that a delimiter for dividing hierarchy differs depending on a system or a manufacturer.

The reason 2 is that, since the input method such as the selection type or the free input is not uniform depending on a system or a manufacturer, the data is also not uniform.

Therefore, since the endoscope terms in the JED form are output in units of one row and one lesion and in connection with the item, the attribute, and the additional information (the attribute “1” and the attribute “2”), the medical support device 10 can accurately extract, as a specific word, a term (character string) associated with the “attribute” from the endoscope-related procedure information, and can perform determination by comparing the extracted term (attribute value) with the standard term.

### Processing of Medical Support Device

Next, processing executed by the medical support device will be described.

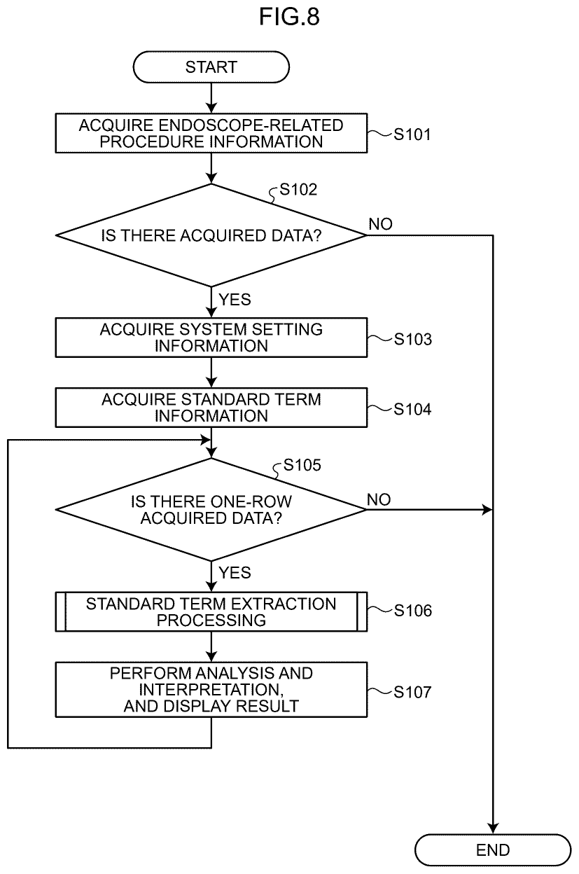

FIG. 8 is a flowchart illustrating an outline of processing executed by the medical support device 10.

As illustrated in FIG. 8, the data acquisition unit 161 acquires the endoscope-related procedure information from the endoscope management device 20 via the communication unit 11 (step S101). For example, the data acquisition unit 161 acquires the endoscope-related procedure information D1 (data in a JED form) described in FIG. 3 from the endoscope management device 20, and records the endoscope-related procedure information D1 in the endoscope-related procedure information DB 140.

Subsequently, the determination unit 162 determines whether or not there is acquired data from the data acquisition unit 161 (step S102). In a case where the determination unit 162 determines that there is acquired data from the data acquisition unit 161 (step S102: Yes), the medical support device 10 proceeds to step S103 to be described later. On the other hand, in a case where the determination unit 162 determines that there is no acquired data from the data acquisition unit 161 (step S102: No), the medical support device 10 ends the processing.

In step S103, the setting unit 163 acquires system setting information recorded in the system setting DB 141. Specifically, the setting unit 163 acquires, from the system setting DB 141, system setting information T3 (refer to FIG. 4) in which a connection character between items, a connection character between attributes, and a connection character between an attribute and additional information in the endoscope-related procedure information are stored as information for dividing and extracting.

Subsequently, the setting unit 163 acquires the standard term information recorded in the standard term accumulation DB 142 (step S104). Specifically, the setting unit 163 acquires the standard term T4 (refer to FIG. 5) and the standard term T5 (refer to FIG. 6) from the standard term accumulation DB 142.

Thereafter, the determination unit 162 determines whether or not the acquired data acquired by the data acquisition unit 161 includes one-row acquired data (step S105). In a case where the determination unit 162 determines that the acquired data acquired by the data acquisition unit 161 includes one-row acquired data (step S105: Yes), the medical support device 10 proceeds to step S106 to be described later. On the other hand, in a case where the determination unit 162 determines that the acquired data acquired by the data acquisition unit 161 does not include one-row acquired data (step S105: No), the medical support device 10 ends the processing.

In step S106, the standard term extraction unit 164 executes standard term extraction processing of extracting a standard term from the one-row acquired data.

### Details of Standard Term Extraction Processing

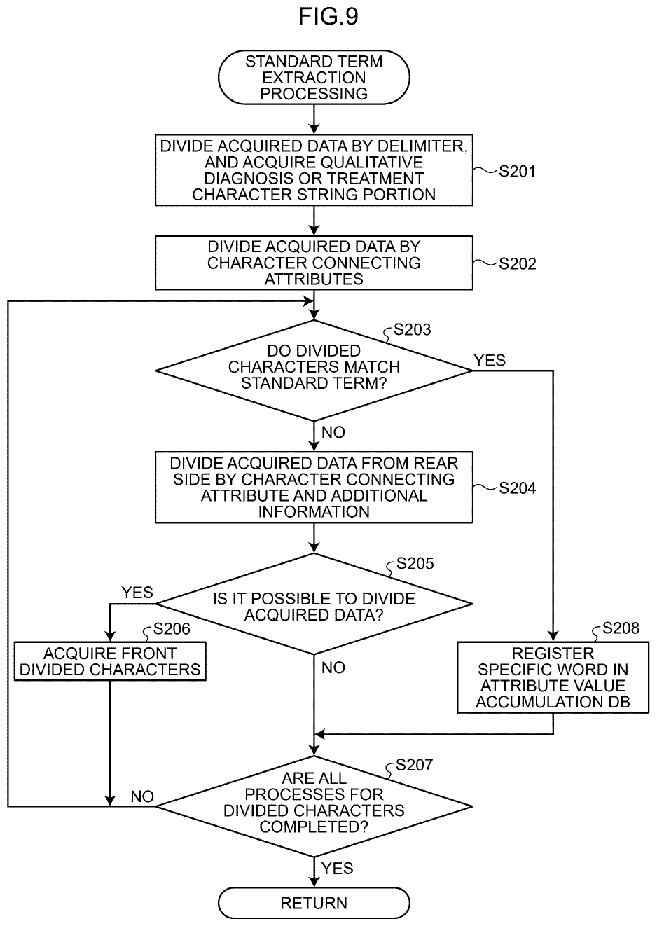

FIG. 9 is a sub-flowchart illustrating an outline of the standard term extraction processing in step S106 of FIG. 8.

As illustrated in FIG. 9, according to the system setting information T3 (refer to FIG. 4) which is set by the setting unit 163, the standard term extraction unit 164 divides the acquired data, which is the endoscope-related procedure information and is acquired by the data acquisition unit 161, by a delimiter character which is a connecting character, and acquires a qualitative diagnosis character string of a specific word (step S201).

Subsequently, the standard term extraction unit 164 divides the acquired data, which is the endoscope-related procedure information and is acquired by the data acquisition unit 161, by a character connecting attributes, according to the system setting information T3 which is set by the setting unit 163 (step S202).

Thereafter, the standard term extraction unit 164 determines whether or not the specific word matches the standard term acquired by the setting unit 163 (step S203). In a case where the standard term extraction unit 164 determines that the specific word matches the standard term acquired by the setting unit 163 (step S203: Yes), the medical support device 10 proceeds to step S208 to be described later. On the other hand, in a case where the standard term extraction unit 164 determines that the specific word does not match the standard term acquired by the setting unit 163 (step S203: No), the medical support device 10 proceeds to step S204 to be described later.

In step S204, the standard term extraction unit 164 divides the acquired data, which is the endoscope-related procedure information and is acquired by the data acquisition unit 161, from a rear side by a character connecting an attribute and additional information, according to the system setting information T3 which is set by the setting unit 163.

In step S205, the standard term extraction unit 164 determines whether or not it is possible to divide the acquired data which is the endoscope-related procedure information from a rear side by a character connecting an attribute and additional information. In a case where the standard term extraction unit 164 determines that it is possible to divide the acquired data which is the endoscope-related procedure information from a rear side by a character connecting an attribute and additional information (step S205: Yes), the medical support device 10 proceeds to step S206. On the other hand, in a case where the standard term extraction unit 164 determines that it is not possible to divide the acquired data which is the endoscope-related procedure information from a rear side by a character connecting an attribute and additional information (step S205: No), the medical support device 10 proceeds to step S207 to be described later.

In step S206, the standard term extraction unit 164 acquires front divided characters as a specific word, from among the characters which are divided and extracted from a rear side. After step S206, the medical support device 10 returns to step S203.

In step S207, the standard term extraction unit 164 determines whether or not all the processes for the specific word generated when performing division in step S202 described above have been completed. In a case where it is determined by the standard term extraction unit 164 that the determination as to whether or not the specific word matches the standard term has been completed for all the specific words extracted by being divided by a character connecting attributes (step S207: Yes), the medical support device 10 returns to the main routine of FIG. 8, and proceeds to step S107. On the other hand, in a case where it is determined by the standard term extraction unit 164 that the determination as to whether or not the specific word matches the standard term has not been completed for all the specific words divided and extracted from a rear side (step S207: No), the medical support device 10 returns to step S203.

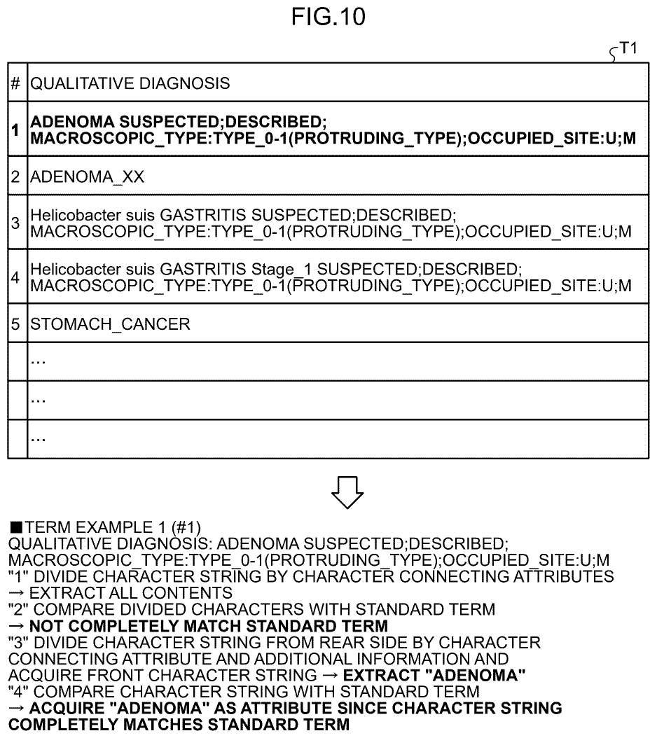

In step S208, the accumulation control unit 165 registers, in the attribute value accumulation DB 143, the specific word determined as matching the standard term in step S203 described above. For example, in the case illustrated in FIG. 10, since the specific word “adenoma” matches the standard term T4 “adenoma”, the standard term extraction unit 164 accumulates and registers, in the attribute value accumulation DB 143, “adenoma” as the attribute of the first line of the qualitative diagnosis text data T1. After step S208, the medical support device 10 proceeds to step S207. Note that, here, the flow for the qualitative diagnosis character string has been described as an example with reference to FIG. 9, but a similar flow is executed for a treatment character string.

### Specific Example of Standard Term Extraction Processing

Here, a specific example of the standard term extraction processing by the standard term extraction unit 164 will be described.

FIG. 10 is a diagram schematically illustrating an example of the standard term extraction processing by the standard term extraction unit 164.

As illustrated in FIG. 10, the standard term extraction unit 164 extracts and divides the first line of the qualitative diagnosis text data T1 as a qualitative diagnosis character string, according to the system setting information T3 (refer to FIG. 4) for division by a delimiter character which is a connecting character and is set by the setting unit 163 (process “1”). In this case, the standard term extraction unit 164 attempts to divide “adenoma suspected;described;macroscopic_type:type_0-1(protruding_type);occupied_site:U;M” by performing division on the basis of the half-width comma “,” of the system setting information T3 for division by a character connecting attributes. However, since the half-width comma “,” is not included in the character string, the standard term extraction unit 164 extracts the original character string “adenoma suspected;described;macroscopic_type:type_0-1(protruding_type);occupied_site:U;M”.

Subsequently, the standard term extraction unit 164 determines whether or not the specific word “adenoma suspected;described;macroscopic_type:type_0-1(protruding_type);occupied_site:U;M” completely matches the standard term T4 (refer to FIG. 5). Since the specific word does not completely match, for example, “adenoma” of the standard term T4, the standard term extraction unit 164 determines that the specific word does not completely match the standard term (process “2”).

Thereafter, the standard term extraction unit 164 performs division on the basis of the half-width space “\s” (half-width spaces) and “ . . . ” in the remark of the system setting information T3, and divides “adenoma suspected;described;macroscopic_type:type_0-1(protruding_type);occupied_site:U;M”, from a rear side by a character connecting an attribute and additional information, into “adenoma” and “suspected;described;macroscopic_type:type_0-1(protruding_type);occupied_site:U;M”. Thereby, the standard term extraction unit 164 extracts “adenoma” and “suspected;described;macroscopic_type:type_0-1(protruding_type);occupied_site:U;M”. Then, the standard term extraction unit 164 acquires front divided characters as a specific word, from among the characters which are divided and extracted from a rear side (process “3”). Specifically, in FIG. 10, the standard term extraction unit 164 acquires “adenoma” that is a specific word in front.

Subsequently, the standard term extraction unit 164 determines whether or not the specific word “adenoma” completely matches the standard term T4 (refer to FIG. 5). For example, since the specific word completely matches “adenoma” of the standard term T4, the standard term extraction unit 164 acquires the specific word “adenoma” as an attribute (process “4”).

The standard term extraction unit 164 determines whether all the processes for the character strings divided by the character connecting attributes have been completed. Specifically, since the character string divided by the character connecting attributes is only “adenoma suspected;described;macroscopic_type:type_0-1(protruding_type);occupied_site:U;M”, it is determined that all the processes for the character strings have been completed.

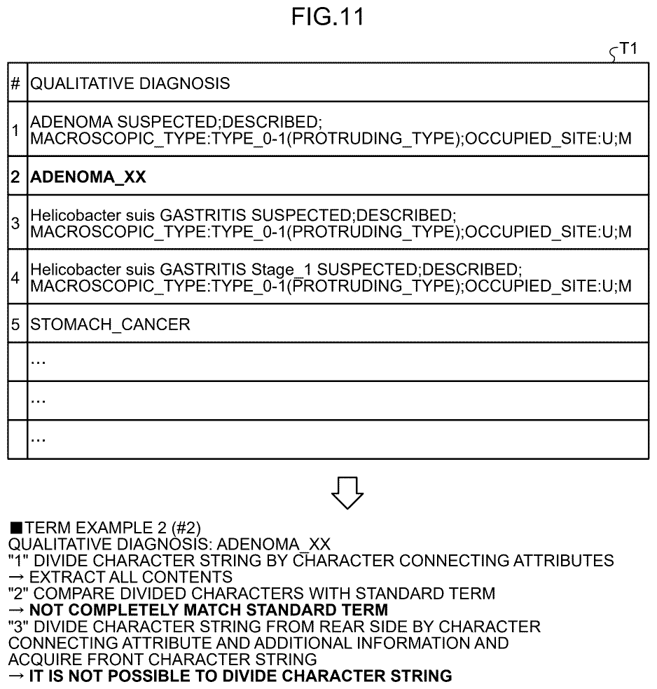

FIG. 11 is a diagram schematically illustrating another example of the standard term extraction processing by the standard term extraction unit 164.

As illustrated in FIG. 11, the standard term extraction unit 164 extracts and divides the second line of the qualitative diagnosis text data T1 as a qualitative diagnosis character string, according to the system setting information T3 (refer to FIG. 4) for division by a delimiter character which is a connecting character and is set by the setting unit 163 (process “1”). In this case, the standard term extraction unit 164 performs division on the basis of the half-width comma “,” of the system setting information T3 for division by a character connecting attributes. In this case, the standard term extraction unit 164 divides and extracts the entire content of “adenoma_XX” as a qualitative diagnosis character string.

Subsequently, the standard term extraction unit 164 determines whether or not the specific word “adenoma_XX” completely matches the standard term T4 (refer to FIG. 5). Since the specific word does not completely match any of the standard terms T4, the standard term extraction unit 164 determines that the specific word does not completely match the standard term (process “2”).

Thereafter, the standard term extraction unit 164 attempts to divide the characters on the basis of the half-width space “\s” and “ . . . ” in the remark of the system setting information T3 from a rear side by a character connecting an attribute and additional information. In this case, the standard term extraction unit 164 determines that it is not possible to divide “adenoma_XX” (process “3”). Then, since the character string divided by a character connecting attributes is only “adenoma_XX”, the standard term extraction unit 164 ends the processing.

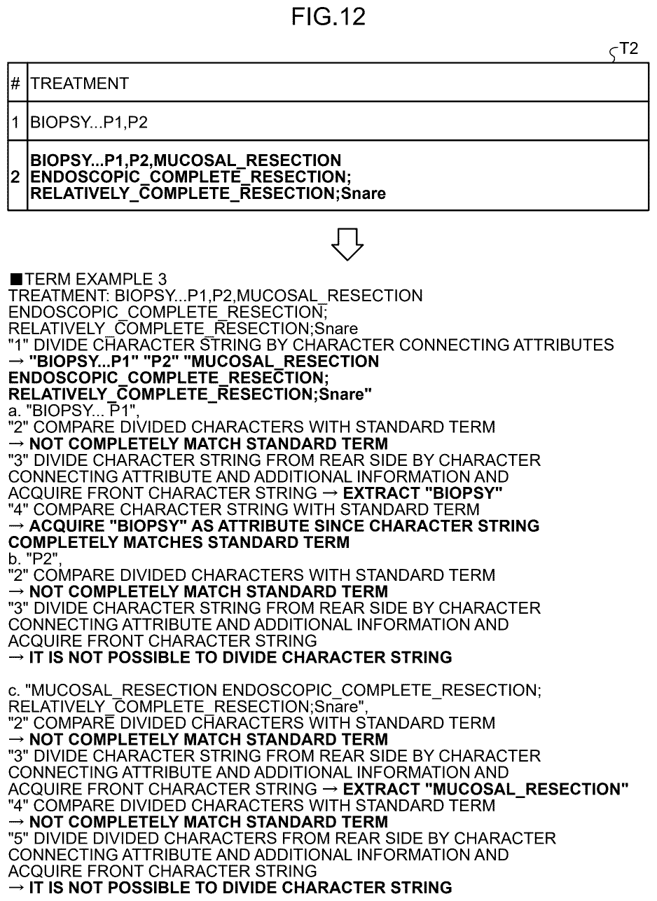

FIG. 12 is a diagram schematically illustrating another example of the standard term extraction processing by the standard term extraction unit 164.

As illustrated in FIG. 12, the standard term extraction unit 164 extracts and divides the second line of the treatment text data T2 as a treatment character string, according to the system setting information T3 (refer to FIG. 4) for division by a delimiter character which is a connecting character and is set by the setting unit 163 (process “1”). In this case, the standard term extraction unit 164 performs division on the basis of the half-width comma “,” of the system setting information T3 for division by a character connecting attributes, and divides “biopsy . . . P1,P2,mucosal_resection endoscopic_complete_resection;relatively_complete_resection;Snare” into “biopsy . . . P1”, “P2”, “mucosal_resection endoscopic_complete_resection;relatively_complete_resection;Snare”. Thus, the standard term extraction unit 164 extracts “biopsy . . . P1”, “P2”, “mucosal_resection endoscopic_complete_resection;relatively_complete_resection;Snare” (process “1”).

Subsequently, the standard term extraction unit 164 performs the following processing on each of “biopsy . . . P1” (a), “P2” (b), and “mucosal_resection endoscopic_complete_resection;relatively_complete_resection;Snare” (c).

First, the case of “biopsy . . . P1” (a) will be described.

The standard term extraction unit 164 determines whether or not the specific word “biopsy . . . P1” completely matches the standard term T5 (treatment) (refer to FIG. 6). Since the specific word “biopsy . . . P1” does not completely match any of the standard terms T5, the standard term extraction unit 164 determines that the specific word does not completely match the standard term (process “2”).

Thereafter, the standard term extraction unit 164 performs division on the basis of the half-width space “\s” (half-width space) and “ . . . ” in the remark of the system setting information T3, and divides “biopsy . . . P1”, from a rear side by a character connecting an attribute and additional information, into “biopsy” and “P1”. Thus, the standard term extraction unit 164 extracts “biopsy” and “P1”. Then, the standard term extraction unit 164 acquires front divided characters as a specific word, from among the characters which are divided and extracted from a rear side (process “3”). Specifically, the standard term extraction unit 164 acquires “biopsy”, which is front divided characters, as a specific word.

Subsequently, the standard term extraction unit 164 determines whether or not the specific word “biopsy” completely matches the standard term T5 (refer to FIG. 6). For example, since the specific word completely matches “biopsy” in the standard term T5, the standard term extraction unit 164 acquires the specific word “biopsy” as an attribute (process “4”).

Next, the case of “P2” (b) will be described.

The standard term extraction unit 164 determines whether or not the specific word “P2” completely matches the standard term T5 (treatment) (refer to FIG. 6). Since the specific word “P2” does not completely match any of the standard terms T5, the standard term extraction unit 164 determines that the specific word does not completely match the standard term (process “2”).

Subsequently, the standard term extraction unit 164 performs division on the basis of the half-width space “\s” and “ . . . ” in the remark of the system setting information T3, and attempts to divide the specific word “P2” from a rear side by a character connecting an attribute and additional information. In this case, the standard term extraction unit 164 determines that it is not possible to divide the specific word “P2” (process “3”).

Since the character string divided by a character connecting attributes is only the specific word “P2”, the standard term extraction unit 164 ends the processing.

Next, the case of “mucosal_resection endoscopic_complete_resection;relatively_complete_resection;Snare” (c) will be described.

The standard term extraction unit 164 determines whether or not the specific word “mucosal_resection endoscopic_complete_resection;relatively_complete_resection;Snare” completely matches the standard term T5 (treatment) (refer to FIG. 6). Since the specific word “mucosal_resection endoscopic_complete_resection;relatively_complete_resection;Snare” does not completely match any of the standard terms T5, the standard term extraction unit 164 determines that the specific word does not completely match the standard term (process “2”).

Subsequently, the standard term extraction unit 164 performs division on the basis of the half-width space “\s” and “ . . . ” in the remark of the system setting information T3, and divides “mucosal_resection endoscopic_complete_resection;relatively_complete_resection;Snare”, from a rear side by a character connecting an attribute and additional information, into “mucosal_resection” and “endoscopic_complete_resection;relatively_complete_resection;Snare”. Thus, the standard term extraction unit 164 extracts “mucosal_resection” and “mucosal_resection” and “endoscopic_complete_resection;relatively_complete_resection;Snare”. Then, the standard term extraction unit 164 acquires front divided characters as a specific word, from among the characters which are divided and extracted from a rear side (process “3”). Specifically, in FIG. 12, the standard term extraction unit 164 acquires “mucosal_resection”, which is front divided characters, as a specific word.

Thereafter, the standard term extraction unit 164 determines whether or not the specific word “mucosal_resection” completely matches any of the standard terms T5 (refer to FIG. 6). The standard term extraction unit 164 determines that the specific word “mucosal_resection” does not completely match any of the standard terms T5 (refer to FIG. 6) (process “4”).

Subsequently, the standard term extraction unit 164 performs division on the basis of the half-width space “\s” and “ . . . ” in the remark of the system setting information T3, and divides “mucosal_resection” from a rear side. Thus, the standard term extraction unit 164 extracts and acquires “mucosal_resection” as a front character string (process “5”). In this case, the standard term extraction unit 164 determines that it is not possible to further divide the front character string “mucosal_resection”.

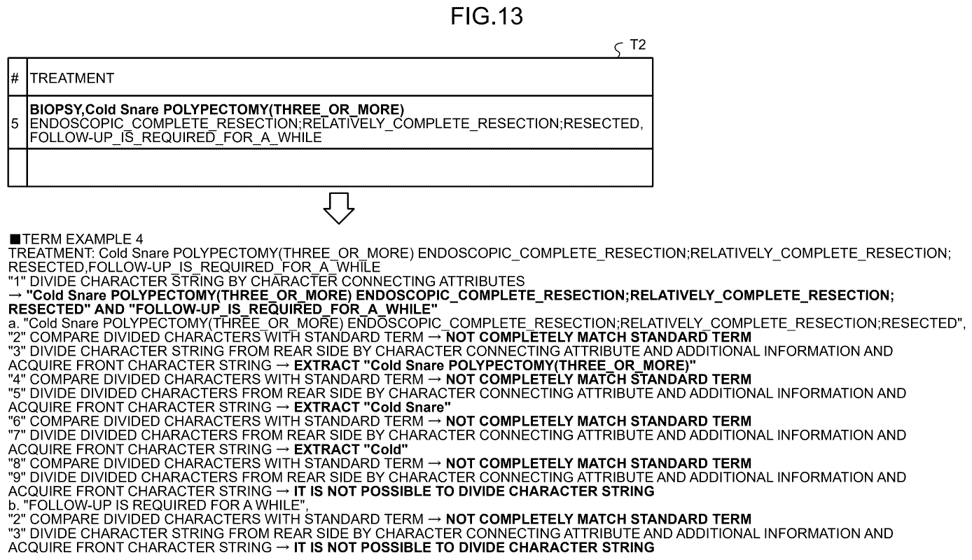

FIG. 13 is a diagram schematically illustrating another example of the standard term extraction processing by the standard term extraction unit 164.

The standard term extraction unit 164 extracts and divides the fifth line of the treatment text data T2 as a treatment character string, according to the half-width comma “,” in the remark of the system setting information T3 (refer to FIG. 4) for division by a delimiter character which is a connecting character and is set by the setting unit 163 (process “1”). In this case, the standard term extraction unit 164 divides “biopsy, Cold Snare polypectomy(three_or_more) endoscopic_complete_resection;relatively_complete_resection;resected,follow-up_is_required_for_a_while” into “biopsy”, “Cold Snare polypectomy(three_or_more) endoscopic_complete_resection;relatively_complete_resection;resected”, and “follow-up_is_required_for_a_while” on the basis of the half-width comma “,” of the system setting information T3 for division by a character connecting attributes. Thus, the standard term extraction unit 164 extracts “biopsy”, “Cold Snare polypectomy(three_or_more) endoscopic_complete_resection;relatively_complete_resection;resected”, and “follow-up_is_required_for_a_while” (process “1”). Since “biopsy” is the same process as the process in FIG. 12 described above, detailed description thereof will be omitted. In the following description, “Cold Snare polypectomy(three_or_more) endoscopic_complete_resection;relatively_complete_resection;resected” and “follow-up_is_required_for_a_while” will be described.

First, the case of “Cold Snare polypectomy(three_or_more) endoscopic_complete_resection;relatively_complete_resection;resected” (a) will be described.

The standard term extraction unit 164 determines whether or not the specific word “Cold Snare polypectomy(three_or_more) endoscopic_complete_resection;relatively_complete_resection;resected” completely matches any of the standard terms T5 (treatment) (refer to FIG. 6). Since the specific word “Cold Snare polypectomy(three_or_more) endoscopic_complete_resection;relatively_complete_resection;resected” does not completely match any of the standard terms T5 (treatment) (refer to FIG. 6), the standard term extraction unit 164 determines that the specific word does not completely match the standard terms (process “2”).

Subsequently, the standard term extraction unit 164 performs division on the basis of the half-width space “\s” and “ . . . ” in the remark of the system setting information T3, and attempts to divide “Cold Snare polypectomy(three_or_more) endoscopic_complete_resection;relatively_complete_resection;resected” from a rear side by a character connecting an attribute and additional information. In this case, the standard term extraction unit 164 extracts a specific word “Cold Snare polypectomy(three_or_more)” (process “3”).

Thereafter, the standard term extraction unit 164 determines whether or not the specific word “Cold Snare polypectomy(three_or_more)” completely matches any of the standard terms T5 (refer to FIG. 6). The standard term extraction unit 164 determines that the specific word “Cold Snare polypectomy(three_or_more)” does not completely match any of the standard terms T5 (process “4”).

Subsequently, the standard term extraction unit 164 performs division on the basis of the half-width space “\s” and “ . . . ” in the remark of the system setting information T3, and divides “Cold Snare polypectomy(three_or_more)”, from a rear side by a character connecting an attribute and additional information, into “Cold Snare” and “polypectomy(three_or_more)”. Thus, the standard term extraction unit 164 extracts “Cold Snare” and “polypectomy(three_or_more)”. Then, the standard term extraction unit 164 acquires front divided characters as a specific word, from among the characters which are divided and extracted from a rear side (process “5”). Specifically, the standard term extraction unit 164 extracts a specific word “Cold Snare” in front (process “5”).

Thereafter, the standard term extraction unit 164 determines whether or not the specific word “Cold Snare” completely matches any of the standard terms T5 (refer to FIG. 6). The standard term extraction unit 164 determines that the specific word “Cold Snare” does not completely match any of the standard terms T5 (process “6”).

Subsequently, the standard term extraction unit 164 performs division on the basis of the half-width space “\s” and “ . . . ” in the remark of the system setting information T3, and divides “Cold Snare”, from a rear side by a character connecting an attribute and additional information, into “Cold” and “Snare”. Thus, the standard term extraction unit 164 extracts “Cold” and “Snare”. Then, the standard term extraction unit 164 acquires front divided characters as a specific word, from among the characters which are divided and extracted from a rear side (process “7”). Specifically, the standard term extraction unit 164 extracts a specific word “Cold” in front (process “7”).

Thereafter, the standard term extraction unit 164 determines whether or not the specific word “Cold” completely matches any of the standard terms T5 (refer to FIG. 6). The standard term extraction unit 164 determines that the specific word “Cold” does not completely match any of the standard terms T5 (process “8”).

Subsequently, the standard term extraction unit 164 performs division on the basis of the half-width space “\s” and “ . . . ” in the remark of the system setting information T3, and attempts to divide “Cold” from a rear side by a character connecting an attribute and additional information. In this case, the standard term extraction unit 164 determines that it is not possible to divide the specific word “Cold” (process “9”).

Since “follow-up_is_required_for_a_while” remains in the divided characters processed in S202 described above (the determination in S207 described above), the case of “follow-up_is_required_for_a_while” will be described below.

The standard term extraction unit 164 determines whether or not “follow-up_is_required_for_a_while” completely matches any of the standard terms T5 (treatment) (refer to FIG. 6). Since “follow-up_is_required_for_a_while”does not completely match any of the standard terms T5, the standard term extraction unit 164 determines that the specific word does not completely match the standard term (process “2”).

Subsequently, the standard term extraction unit 164 performs division on the basis of the half-width space “\s” and “ . . . ” in the remark of the system setting information T3, and attempts to divide “follow-up_is_required_for_a_while” from a rear side by a character connecting an attribute and additional information. In this case, the standard term extraction unit 164 determines that it is not possible to divide the specific word “follow-up_is_required_for_a_while” (process “3”).

Thereafter, the character string divided by the character connecting attributes has reached the specific word “follow-up_is_required_for_a_while”, and processes on all the specific words (divided characters) divided in step S202, specifically, “biopsy”, “Cold Snare polypectomy(three_or_more) endoscopic_complete_resection;relatively_complete_resection;resected”, and “follow-up_is_required_for_a_while” are ended. Thus, the standard term extraction unit 164 ends the standard term extraction processing.

Returning to FIG. 8, description of step S107 and subsequent steps will be continued.

In step S107, the analysis/interpretation unit 166 performs analysis/interpretation on the basis of the attribute value accumulation data accumulated in the attribute value accumulation DB 143, and displays an analysis/interpretation result obtained by the analysis/interpretation on the display unit 12.

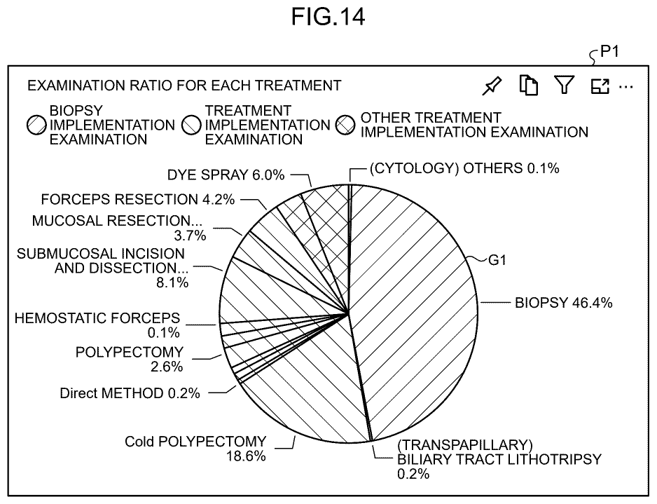

FIG. 14 is a diagram illustrating an example of an analysis result analyzed by the analysis/interpretation unit 166.

As illustrated in FIG. 14, the analysis/interpretation unit 166 analyzes the treatment information included in the attribute value accumulation data on the basis of the attribute value accumulation data accumulated in the attribute value accumulation DB 143, analyzes an examination ratio with attribute items in a second layer, and displays an interpretation result P1 on the display unit 12. Thereby, a user such as a doctor can intuitively recognize the examination ratio.

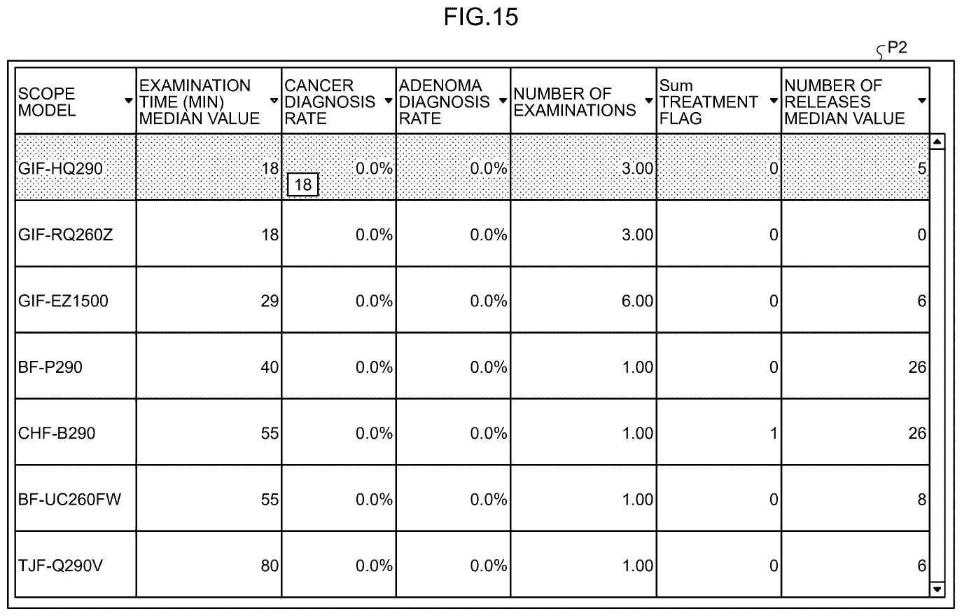

FIG. 15 is a diagram illustrating another example of an interpretation result interpreted by the analysis/interpretation unit 166.

As illustrated in FIG. 15, the analysis/interpretation unit 166 interprets the qualitative diagnosis information included in the attribute value accumulation data on the basis of the attribute value accumulation data accumulated in the attribute value accumulation DB 143, further classifies “cancer” and “adenoma” from the extracted attribute items of the second layer, and displays an interpretation result P2 obtained by interpreting the diagnosis rate (number of diagnosed examinations/total number of examinations) on the display unit 12. Thereby, a user such as a doctor can intuitively recognize the diagnosis rate.

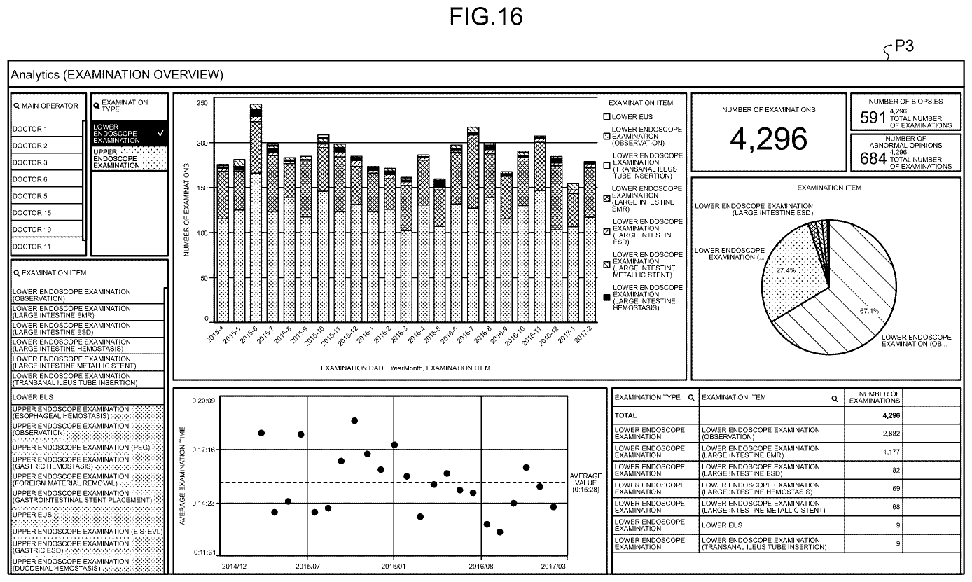

FIG. 16 is a diagram illustrating another example of an interpretation result interpreted by the analysis/interpretation unit 166.

As illustrated in FIG. 16, the analysis/interpretation unit 166 displays, on the display unit 12, an interpretation result P3 obtained by analyzing and interpreting various types of information including, for each examination item, the number of examinations, the number of biopsies, opinions, and the like, on the basis of the endoscope-related procedure information acquired from the endoscope management device 20. Thereby, a user such as a doctor can intuitively recognize the number of examinations.

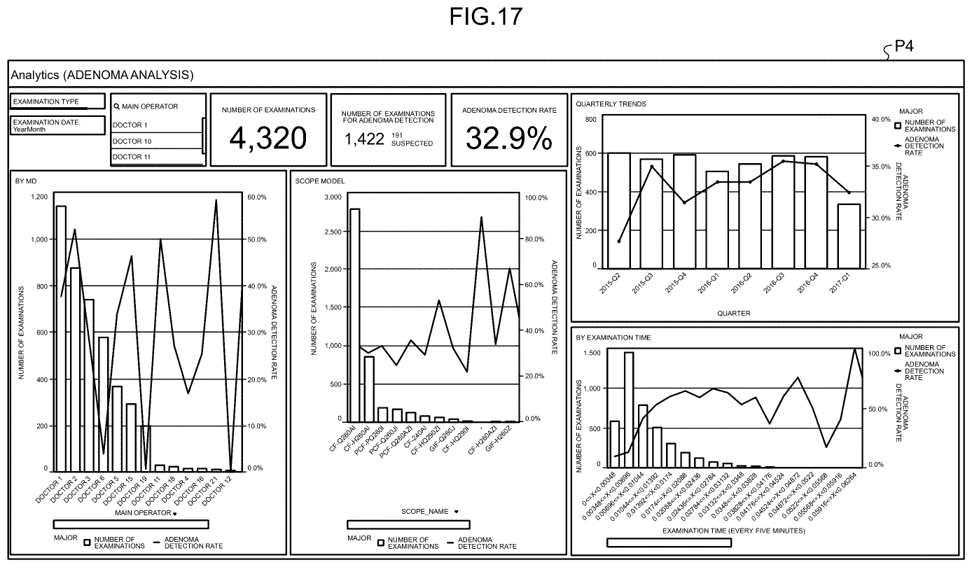

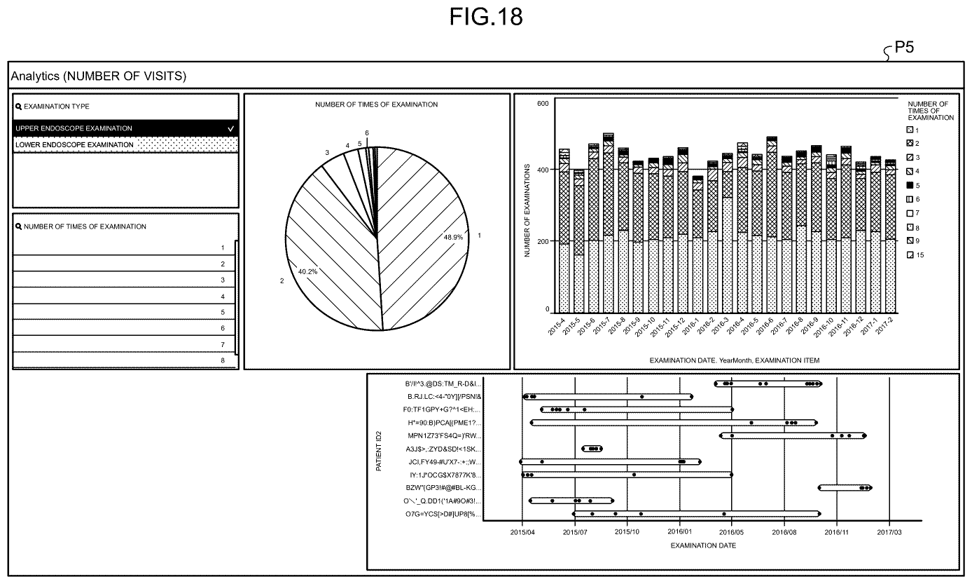

FIG. 17 is a diagram illustrating another example of an interpretation result interpreted by the analysis/interpretation unit 166.

As illustrated in FIG. 17, the analysis/interpretation unit 166 displays, on the display unit 12, an interpretation result P4 related to adenoma analysis on the basis of the endoscope-related procedure information acquired from the endoscope management device 20. Thereby, the user such as a doctor can intuitively recognize the number of examinations according to the attribute.

FIG. 18 is a diagram illustrating another example of an interpretation result interpreted by the analysis/interpretation unit 166.

As illustrated in FIG. 18, the analysis/interpretation unit 166 displays, on the display unit 12, an interpretation result P5 obtained by analyzing and interpreting various types of information including, for each examination item, the number of examinations, the number of biopsies, opinions, and the like on the basis of the endoscope-related procedure information acquired from the endoscope management device 20. Thereby, a user such as a doctor can intuitively recognize the number of examinations. After step S108, the medical support device 10 returns to step S105.

According to the first embodiment described above, the accumulation control unit 165 registers, as the attribute value, the specific word which is determined as matching the standard term extracted by the standard term extraction unit 164 in the attribute value accumulation DB 143. Thus, it is possible to accurately extract the term to be used for analysis.

In addition, according to the first embodiment, the analysis/interpretation unit 166 performs analysis/interpretation on the basis of the attribute value accumulation data accumulated by the attribute value accumulation DB 143, and displays the result on the display unit 12. Thus, the user such as a doctor can intuitively recognize the examination ratio.

Note that, in the first embodiment, the specific word which is determined as the attribute value by the analysis is accumulated in the attribute value accumulation DB 143, but the present embodiment is not limited thereto. In the first embodiment, for example, the specific word which is determined as the attribute value may be output to another system or server without being accumulated in the present device, and may be used for processing, analysis, and the like.

Further, in the first embodiment, the standard term extraction unit 164 divides the endoscope-related procedure information from a rear side by a character connecting an attribute and additional information according to the standard term T4 (refer to FIG. 5) or the standard term T5 which is set by the setting unit 163, but the present disclosure is not limited thereto. For example, a specific word that is an evaluation target may be acquired by sequentially removing information added to the rear side. Specifically, the standard term extraction unit 164 may divide all the pieces of endoscope-related procedure information, and sequentially combine the evaluation targets.

Further, in the first embodiment, step S107 of performing analysis/interpretation and display is connected in series with the data processing, but the present disclosure is not limited thereto. For example, after the data acquisition processing is completed as the flow returning from S106 to S105, the analysis/interpretation processing in step S107 may be performed on data accumulated in the attribute value accumulation DB 143 as another flowchart.

### Second Embodiment

Next, a second embodiment will be described. In the first embodiment described above, it is determined whether or not the specific word extracted by the standard term extraction unit 164 matches the standard term. On the other hand, in a second embodiment, in a case where the specific word does not match the standard term, it is determined whether or not the specific word is a facility-specific term. In a case where the specific word is a facility-specific term, the specific word is determined and accumulated as a facility-specific term. Therefore, in the following description, a functional configuration of a medical support device according to a second embodiment will be described, and then processing executed by the medical support device according to a second embodiment will be described. Note that the same components as those of the medical support device 10 according to the first embodiment described above are denoted by the same reference numerals, and a detailed description thereof will be omitted.

### Functional Configuration of Medical Support Device

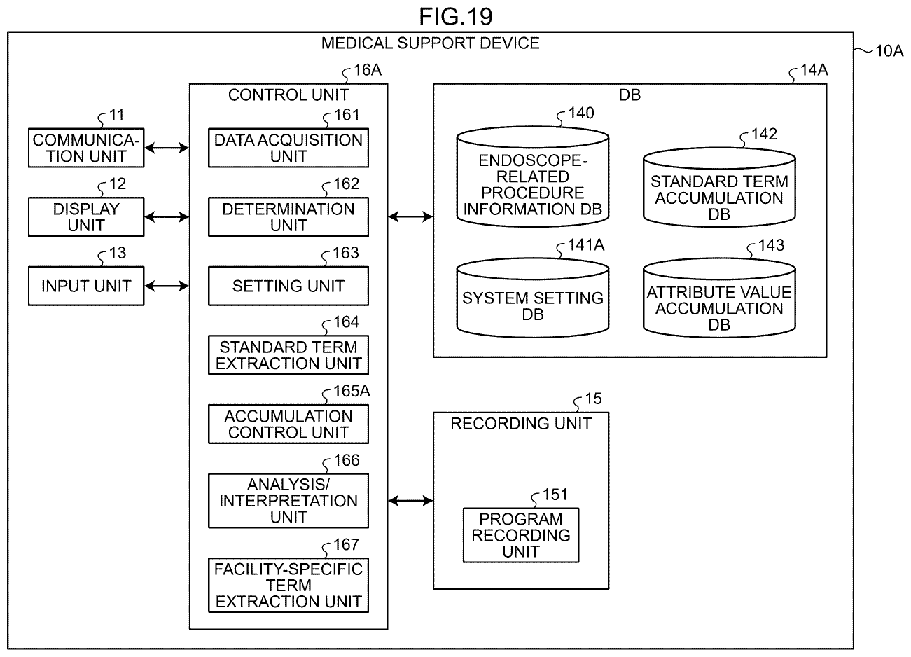

FIG. 19 is a block diagram illustrating a functional configuration of a medical support device according to a second embodiment.

The medical support device 10A illustrated in FIG. 19 includes a DB 14A and a control unit 16A, instead of the DB 14 and the control unit 16 of the medical support device 10 according to the first embodiment described above.

The DB 14A records various types of information. The DB 14A is configured using an HDD, an SSD, or the like. The DB 14A includes a system setting DB 141A, instead of the system setting DB 141 of the DB 14 according to the first embodiment described above.

The system setting DB 141A records system setting information in which a character division method, an attribute, and additional information that are related to a specific word and are extracted from the endoscope-related procedure information are associated.

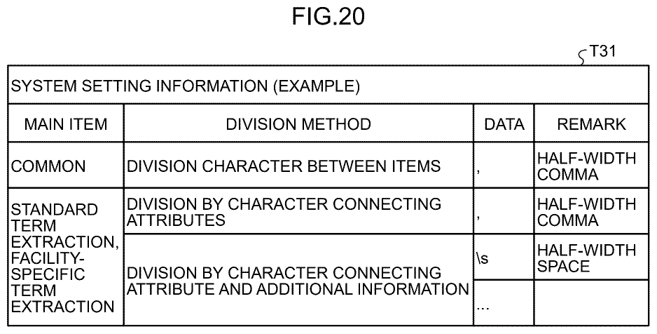

FIG. 20 is a diagram illustrating an example of system setting information recorded in the system setting DB 141A.

The system setting information T31 illustrated in FIG. 20 stores, as information for dividing and extracting, a connection character between items, a connection character between attributes, and a connection character between an attribute and additional information in the endoscope-related procedure information. Specifically, the system setting information T31 stores the contents of a division method, data, and remark in association with main items. For example, a division method “division character between items”, data “,”, and remark “half-width comma” are stored in association with a main item “common” of the system setting information T31. In addition, division methods “division by character connecting attributes” and “division by character connecting attribute and additional information”, data “,”, “,”, “\s”, and “ . . . ”, and remarks “half-width comma”, “half-width comma”, and “half-width space” are stored in association with a main item “standard term extraction, facility-specific term extraction” of the system setting information T31.

The control unit 16A corresponds to a processor according to the present disclosure. The control unit 16A is implemented by using a processor including hardware such as a GPU or a CPU and a memory which is a temporary storage area used by the processor. In addition, the control unit 16A comprehensively controls each unit included in the medical support device 10A. The control unit 16A includes an accumulation control unit 165A, instead of the accumulation control unit 165. Further, the control unit 16A further includes a facility-specific term extraction unit 167 in addition to the configuration of the control unit 16 according to the first embodiment described above.

The accumulation control unit 165A registers, as an attribute value, the specific word which is determined as matching the standard term by the standard term extraction unit 164, in the attribute value accumulation DB 143. In addition, the accumulation control unit 165A registers, as the attribute value, the specific word which is determined as meeting a facility-specific term extraction condition by the facility-specific term extraction unit 167, in the standard term accumulation DB 142 and the attribute value accumulation DB 143.

The facility-specific term extraction unit 167 extracts and acquires divided characters which are specific words of the facility-specific terms by dividing the endoscope-related procedure information by using the system setting information T31.

Meanwhile, as described in the first embodiment, there are a plurality of manufacturers with systems capable of outputting data in the JED form, and the output form, the term selection, and the term input are different for each system. For this reason, in the related art, in a case of a hierarchical structure including items, attributes, and additional information (attribute “1” and attribute “2”), which are appropriately set in the JED form, it is difficult to recognize which location needs to be set as an analysis target (extraction target). Further, in addition to the reason 1 and the reason 2 described above, the reason 3 is that it is desired to further count and analyze facility-specific terms.

The reason 3 is that, since names of the terms are changed according to the preferences of users such as doctors, names of the terms do not always completely match with names of additional information (items, attributes, attribute “1” and attribute “2”) defined in the JED form.

Therefore, since the endoscope terms in the JED form are output in units of one row and one lesion and in connection with the item, the attribute, and the additional information (the attribute “1” and the attribute “2”), even in a case where the term does not match the standard term, the medical support device 10A determines a term (attribute value) of a character string that is an extracted specific word, as a facility-specific term, and accumulates and registers the term. Thereby, the medical support device 10A can further contribute to improvement of a quality of medical care and provision of best medical care to patients.

### Processing of Medical Support Device

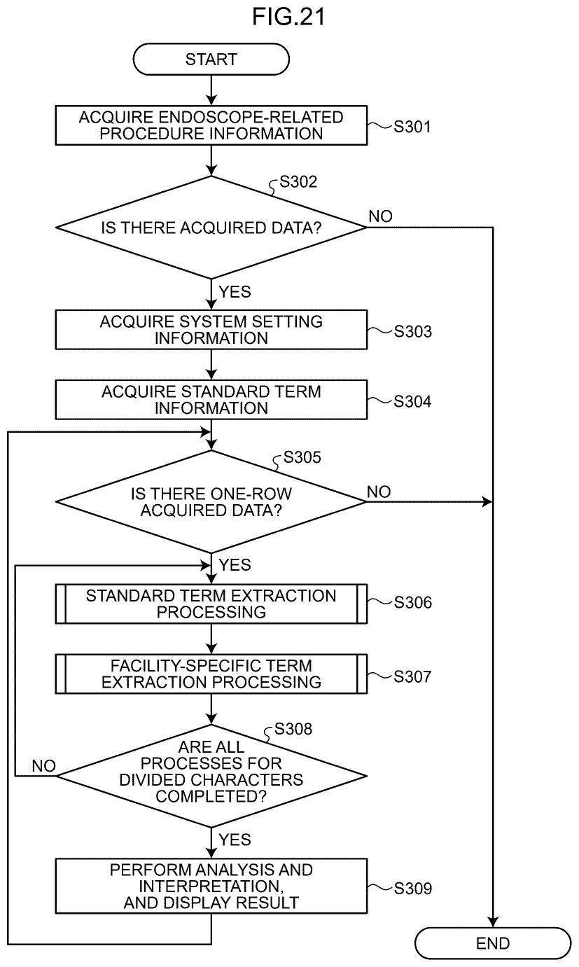

Next, processing executed by the medical support device 10A will be described. FIG. 21 is a flowchart illustrating an outline of processing executed by the medical support device 10A. In FIG. 21, step S301 to step S305 and step S309 respectively correspond to step S101 to step S105 and step S107 in FIG. 8 described above, and step S306 to step S308 are different. Therefore, in the following description, step S306 to step S308 will be described.

In step S306, the standard term extraction unit 164 executes standard term extraction processing of extracting a standard term from the one-row acquired data.

### Details of Standard Term Extraction Processing

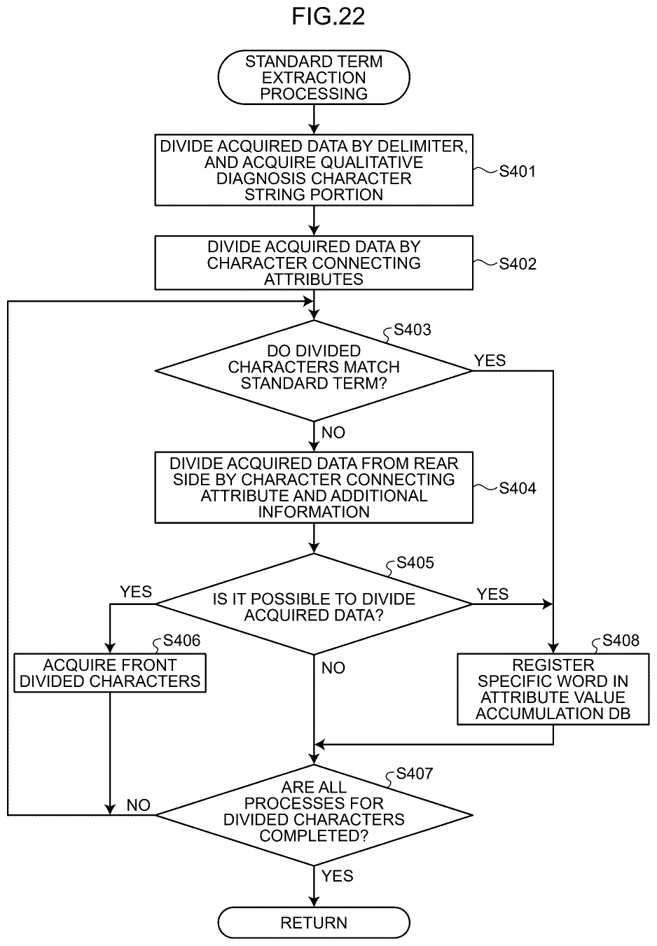

FIG. 22 is a sub-flowchart illustrating an outline of the standard term extraction processing in step S306 of FIG. 21.

In FIG. 22, step S401 to step S408 respectively perform the same processes as step S201 to step S208 in FIG. 9 described above, and the processing to be performed on the acquired data which is the endoscope-related procedure information is different from the processing of the first embodiment described above. Therefore, in the following description, a specific example of the standard term extraction processing by the standard term extraction unit 164 will be described.

### Specific Example of Standard Term Extraction Processing

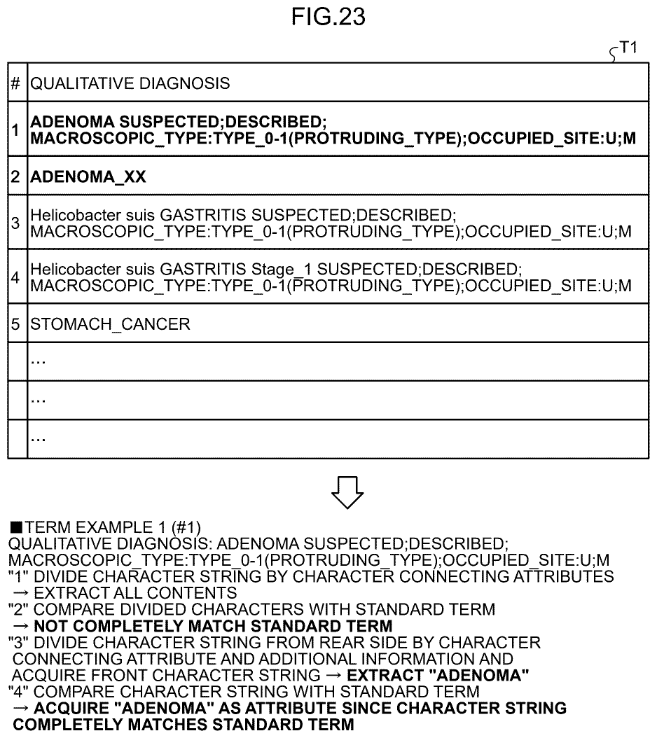

FIG. 23 is a diagram schematically illustrating an example of the standard term extraction processing by the standard term extraction unit 164.

As illustrated in FIG. 23, the standard term extraction unit 164 performs the same processing of process “1” to process “4” as the processing illustrated in FIG. 10, on the first line of the qualitative diagnosis text data T1, according to the system setting information T3 (refer to FIG. 4) for division by a delimiter character which is a connecting character and is set by the setting unit 163. In the case illustrated in FIG. 23, the standard term extraction unit 164 determines whether or not the specific word “adenoma” completely matches the standard term T4 (refer to FIG. 5), and acquires the specific word “adenoma” as an attribute since the specific word completely matches “adenoma” of the standard term T4.

The standard term extraction unit 164 determines whether all the processes for the character strings divided by the character connecting attributes have been completed. Specifically, since the character string divided by the character connecting attributes is only “adenoma suspected;described;macroscopic_type:type_0-1(protruding_type);occupied_site:U+M”, it is determined that all the processes for the character strings have been completed.

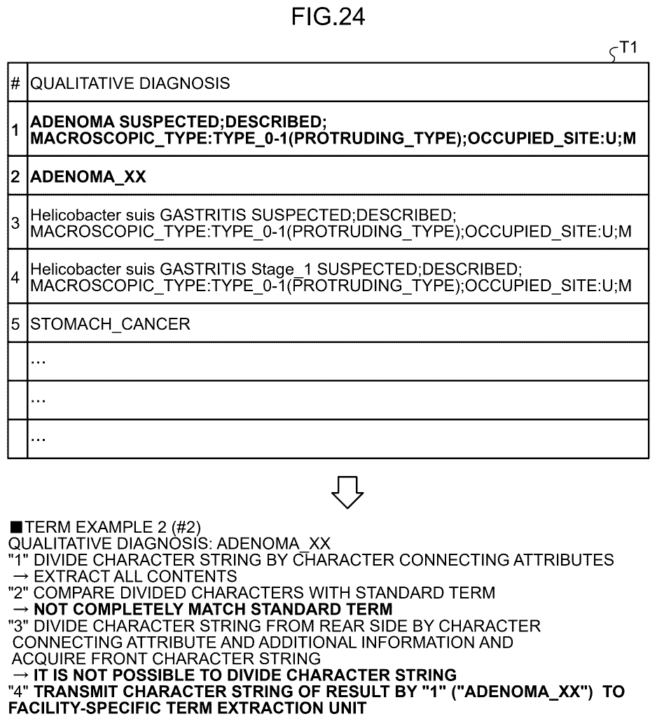

FIG. 24 is a diagram schematically illustrating another example of the standard term extraction processing by the standard term extraction unit 164.

As illustrated in FIG. 24, the standard term extraction unit 164 performs the same processing of process “1” to process “3” as the processing illustrated in FIG. 11, on the second line of the qualitative diagnosis text data T1, according to the system setting information T3 (refer to FIG. 4) for division by a delimiter character which is a connecting character and is set by the setting unit 163. In addition, since the specific word “adenoma_XX” does not completely match any of the standard terms T4 (refer to FIG. 5), the standard term extraction unit 164 determines that it is not possible to divide the specific word, and outputs a character string “adenoma_XX” that is a result of the process “1” to the facility-specific term extraction unit 167 to be described later (processing of “process 4”). Then, since the character string divided by a character connecting attributes is only “adenoma_XX”, the standard term extraction unit 164 ends the processing.

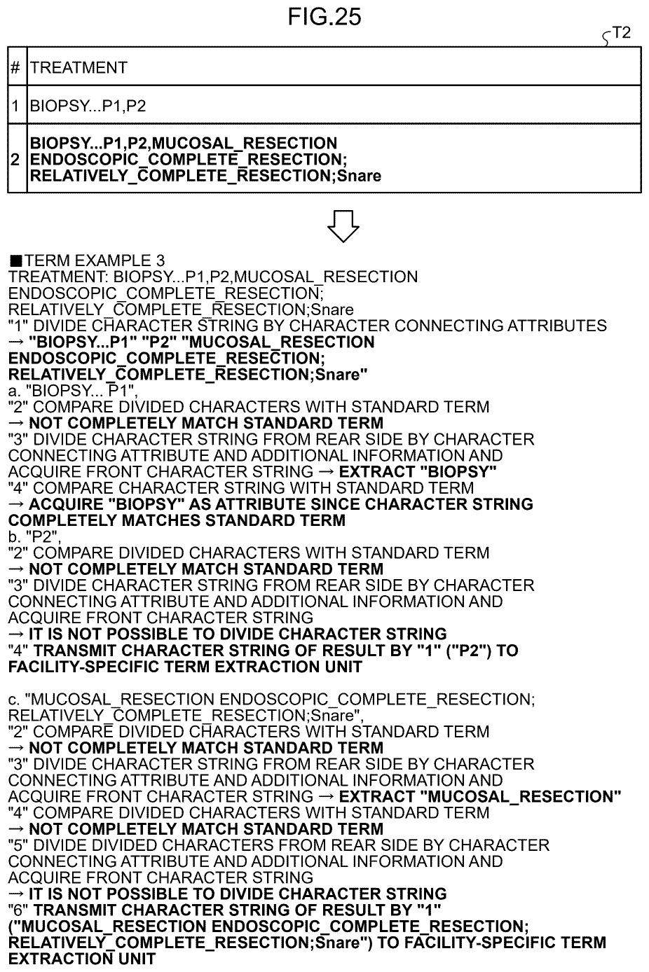

FIG. 25 is a diagram schematically illustrating another example of the standard term extraction processing by the standard term extraction unit 164.

As illustrated in FIG. 25, the standard term extraction unit 164 extracts and divides the second line of the treatment text data T2 as a treatment character string by performing the same processing of process “1” as the processing illustrated in FIG. 12, according to the system setting information T3 (refer to FIG. 4) for division by a delimiter character which is a connecting character and is set by the setting unit 163. In this case, the standard term extraction unit 164 divides “biopsy . . . P1,P2,mucosal_resection endoscopic_complete_resection;relatively_complete_resection;Snare” on the basis of the half-width comma “,” of the system setting information T3 for division by a character connecting attributes. Thus, the standard term extraction unit 164 extracts “biopsy . . . P1”, “P2”, “mucosal_resection endoscopic_complete_resection;relatively_complete_resection;Snare”.

Subsequently, the standard term extraction unit 164 performs the following processing on each of “biopsy . . . P1” (a), “P2” (b), and “mucosal_resection endoscopic_complete_resection;relatively_complete_resection;Snare” (c).

First, the case of “biopsy . . . P1” (a) will be described.

The standard term extraction unit 164 performs same processing of process “2” to process “4” as the processing in FIG. 12 described above, on the specific word “biopsy . . . P1” (a). In the case illustrated in FIG. 25, the standard term extraction unit 164 acquires the front divided characters as a specific word from among the characters which are divided and extracted from a rear side, determines whether or not the acquired specific word “biopsy” completely matches the standard term T5 (refer to FIG. 6), and acquires the specific word “biopsy” as an attribute since the specific word completely matches “biopsy” of the standard term T5.

Next, the case of “P2” (b) will be described.

The standard term extraction unit 164 performs same processing of process “2” and process “3” as the processing in FIG. 12 described above, on the specific word “P2”. In addition, since the specific word “P2” does not completely match any of the standard terms T5 (refer to FIG. 5) and it is not possible to divide the specific word “P2” even in a case where the standard term extraction unit 164 attempts to divide the specific word from a rear side by a character connecting an attribute and additional information, the standard term extraction unit 164 outputs the character string “P2” that is a result of the process “1” to the facility-specific term extraction unit 167 to be described later (process “4”).

Next, the case of “mucosal_resection endoscopic_complete_resection;relatively_complete_resection;Snare” (c) will be described.

The standard term extraction unit 164 performs the same processing of process “2” to process “5” as the processing in FIG. 12 described above, on the specific word “mucosal_resection endoscopic_complete_resection;relatively_complete_resection;Snare”. In addition, since the specific word “mucosal resection” does not completely match any of the standard terms T5 (refer to FIG. 5) and it is not possible to divide the specific word “mucosal_resection” even in a case where the standard term extraction unit 164 attempts to divide the specific word from a rear side by a character connecting an attribute and additional information, the standard term extraction unit 164 outputs the character string “mucosal_resection endoscopic_complete_resection;relatively_complete_resection;Snare” that is a result of the process “1” to the facility-specific term extraction unit 167 to be described later (process “6”).

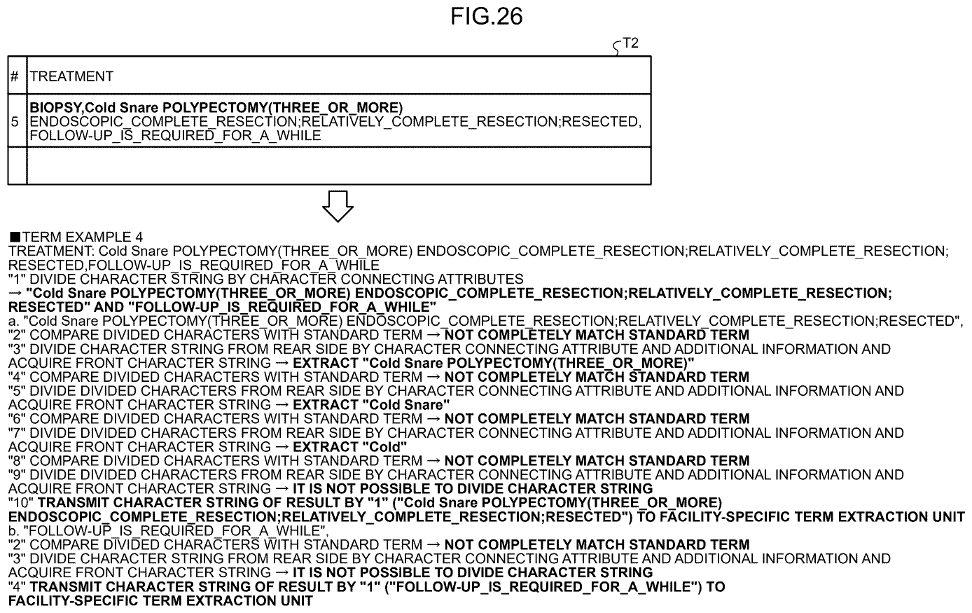

FIG. 26 is a diagram schematically illustrating another example of the standard term extraction processing by the standard term extraction unit 164.

The standard term extraction unit 164 extracts and divides the fifth line of the treatment text data T2 as a treatment character string, according to the half-width comma “,” in the remark of the system setting information T3 (refer to FIG. 4) for division by a delimiter character which is a connecting character and is set by the setting unit 163 (process “1”). In this case, the standard term extraction unit 164 divides “biopsy, Cold Snare polypectomy(three_or_more) endoscopic_complete_resection;relatively_complete_resection;resected, follow-up_is_required_for_a_while” into “biopsy”, “Cold Snare polypectomy(three_or_more) endoscopic_complete_resection;relatively_complete_resection;resected”, and “follow-up_is_required_for_a_while” on the basis of the half-width comma “,” of the system setting information T3 for division by a character connecting attributes. Thus, the standard term extraction unit 164 extracts “biopsy”, “Cold Snare polypectomy(three_or_more) endoscopic_complete_resection;relatively_complete_resection;resected”, and “follow-up_is_required_for_a_while” (process “1”). Since “biopsy” is the same process as the process in FIG. 12 described above, detailed description thereof will be omitted. In the following description, “Cold Snare polypectomy(three_or_more) endoscopic_complete_resection;relatively_complete_resection;resected” and “follow-up_is_required_for_a_while” will be described.

First, the case of “Cold Snare polypectomy(three_or_more) endoscopic_complete_resection;relatively_complete_resection;resected” (a) will be described.

As illustrated in FIG. 26, the standard term extraction unit 164 performs the same processing of process “2” to process “9” as the processing in FIG. 13 described above, on the specific word “Cold Snare polypectomy(three_or_more) endoscopic_complete_resection;relatively_complete_resection;resected”. In addition, the standard term extraction unit 164 performs division on the basis of the half-width space “\s” and “ . . . ” in the remark of the system setting information T3, and attempts to divide the specific word “Cold” from a rear side by a character connecting an attribute and additional information. In this case, since it is not possible to divide the specific word, the standard term extraction unit 164 outputs a character string “Cold Snare polypectomy(three_or_more) endoscopic_complete_resection;relatively_complete_resection;resected” that is a result of process “1” to the facility-specific term extraction unit 167 to be described later (process “10”).

Next, the case of “follow-up_is_required_for_a_while” will be described.

Similarly, the standard term extraction unit 164 performs the same processing of process “2” and process “3” as the processing in FIG. 13 described above, on the specific word “follow-up_is_required_for_a_while”. Then, the standard term extraction unit 164 performs division on the basis of the half-width space “\s” and “ . . . ” in the remark of the system setting information T3, and attempts to divide “follow-up_is_required_for_a_while” from a rear side by a character connecting an attribute and additional information. In this case, since it is not possible to divide the character string, the standard term extraction unit 164 outputs a character string “follow-up_is_required_for_a_while” that is a result of process “1” to the facility-specific term extraction unit 167 to be described later (process “4”).

Returning to FIG. 21, description of step S307 and subsequent steps will be continued.

In step S307, the facility-specific term extraction unit 167 executes facility-specific term extraction processing of extracting a facility-specific term, on the acquired data (specific word) which is acquired by the data acquisition unit 161 and is divided by the standard term extraction unit 164.

### Facility-Specific Term Extraction Processing

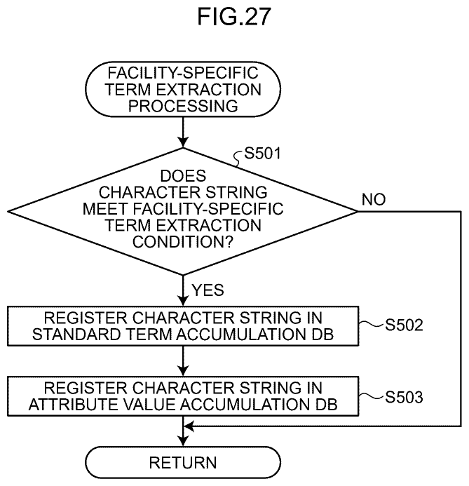

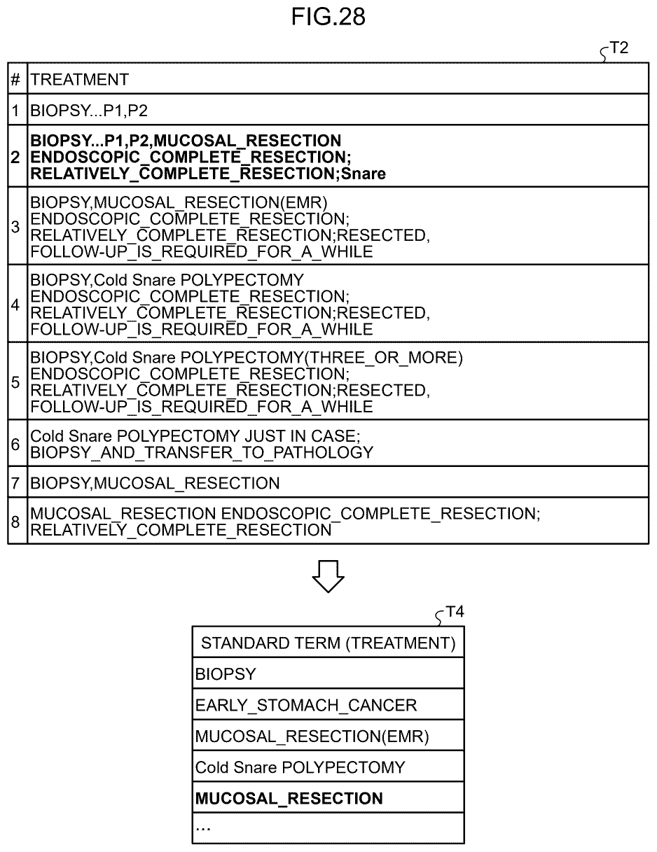

FIG. 27 is a sub-flowchart illustrating an outline of facility-specific term extraction processing in step S307 of FIG. 21. FIG. 28 is a diagram schematically illustrating an example of facility-specific term extraction processing by the facility-specific term extraction unit 167.

As illustrated in FIG. 27, the facility-specific term extraction unit 167 determines whether or not the specific word which is input from the standard term extraction unit 164 meets a facility-specific term extraction condition, according to the system setting information T31 which is set by the setting unit 163 (step S501). Specifically, as illustrated in FIG. 28, the facility-specific term extraction unit 167 performs division on the basis of the half-width comma “,” and “ . . . ” of the system setting information T31, divides the specific word “P2” and the specific word “mucosal_resection endoscopic_complete_resection;relatively_complete_resection;Snare”, which are divided and input by the standard term extraction unit 164, from a rear side by a character connecting an attribute and additional information, extracts and acquires the specific word “mucosal_resection” as a front character string, and determines whether or not “mucosal_resection” meets the facility-specific term extraction condition. In a case where the facility-specific term extraction unit 167 determines that the specific word which is input from the standard term extraction unit 164 meets the facility-specific term extraction condition (step S501: Yes), the medical support device 10A proceeds to step S502 to be described later. On the other hand, in a case where the facility-specific term extraction unit 167 determines that the specific word which is input from the standard term extraction unit 164 does not meet the facility-specific term extraction condition (step S501: No), the medical support device 10A returns to the main routine of FIG. 21, and proceeds to step S308.

In step S502, the accumulation control unit 165 registers the character string which is acquired in step S501 by the facility-specific term extraction unit 167, in the standard term accumulation DB 142. Specifically, as illustrated in FIG. 28, the accumulation control unit 165 registers “mucosal_resection” as a new standard term (treatment), in the standard term T4 recorded by the standard term accumulation DB 142.

Subsequently, the accumulation control unit 165A registers the character string acquired by the facility-specific term extraction unit 167 in step S501, in the attribute value accumulation DB 143 (step S503).

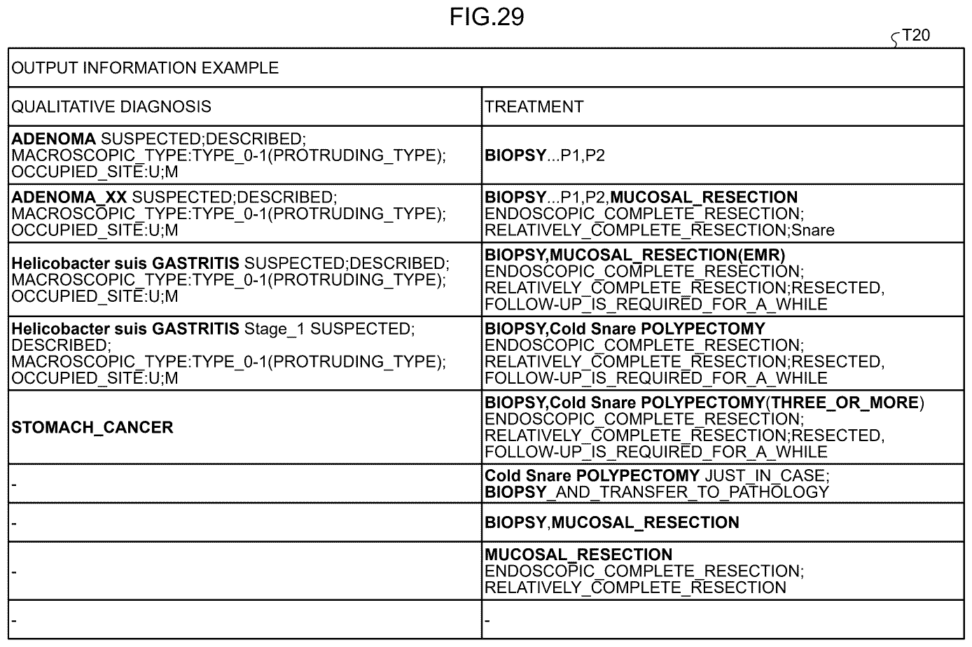

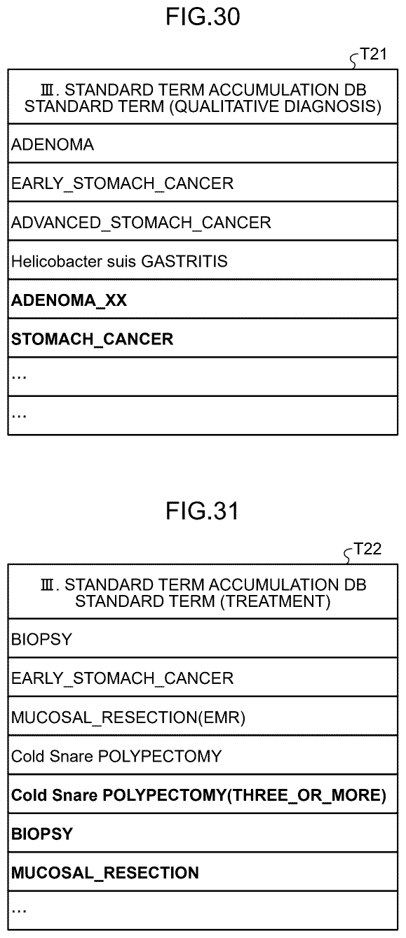

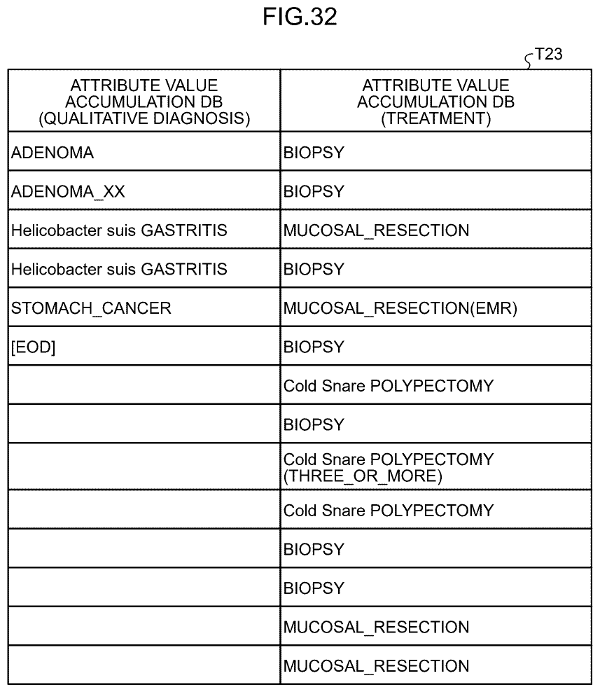

FIG. 29 is a diagram illustrating an example of data which is input when an attribute value of the acquired data is accumulated in the attribute value accumulation DB 143 (a bold portion is accumulated as an attribute value). FIG. 30 is a diagram illustrating an example of a standard term (qualitative diagnosis) accumulated in the standard term accumulation DB 142. FIG. 31 is a diagram illustrating an example of a standard term (treatment) accumulated in the standard term accumulation DB 142. FIG. 32 is a diagram illustrating an example of qualitative diagnosis and treatment of attribute values accumulated in the attribute value accumulation DB 143.

As illustrated in the accumulation data table T20 of FIG. 29, the standard term (qualitative diagnosis) data table T21 of FIG. 30, the standard term (treatment) data table T22 of FIG. 31, and the attribute value table T23 of FIG. 32 for each of qualitative diagnosis and treatment of the attribute values, the accumulation control unit 165 accumulates the attribute values which are extracted by the standard term extraction unit 164 and the facility-specific term extraction unit 167. After step S503, the medical support device 10A returns to the main routine of FIG. 21, and proceeds to step S308.

Returning to FIG. 21, description of step S308 and subsequent steps will be continued.

In step S308, the facility-specific term extraction unit 167 determines whether or not all the processes for the specific word generated when performing division have been completed. In a case where the facility-specific term extraction unit 167 determines that all the processes for the specific word generated when performing division have been completed (step S308: Yes), the medical support device 10A proceeds to S309. On the other hand, in a case where the facility-specific term extraction unit 167 determines that all the processes for the specific word generated when performing division have not been completed (step S308: No), the medical support device 10A returns to step S306.

According to the second embodiment described above, in addition to the effects of the first embodiment described above, since the accumulation control unit 165A accumulates the attribute value which is extracted by the facility-specific term extraction unit 167, even in a case where a specific word different from the standard term is extracted, the specific word can be accumulated as the facility-specific term, and as a result, it is possible to accurately extract the term to be used for analysis.

Note that, in the present embodiment, the system setting information T3 (refer to FIG. 4) and the system setting information T31 (refer to FIG. 20) are treated as independent information, but in an actual configuration, generally, there is no need to use different dividing characters between the standard term extraction unit 164 and the facility-specific term extraction unit 167. Therefore, these pieces of information may be the same information. In that case, equivalent effects can be obtained with a simpler configuration.

### Third Embodiment

Next, a third embodiment of the present disclosure will be described. In the first and second embodiments described above, the data in the JED form that is the endoscope-related procedure information is divided using the preset system setting information, regardless of the type of the system. On the other hand, in the third embodiment, a value (identifier) for identifying the system is stored in the data in the JED form, and one of a plurality of pieces of system setting information which are set in advance is selected on the basis of the value. Therefore, in the following description, a configuration of a medical support device according to the third embodiment will be described, and then processing executed by the medical support device according to the third embodiment will be described. Note that the same components as those of the medical support devices 10 and 10A according to the first and second embodiments described above are denoted by the same reference numerals, and a detailed description thereof will be omitted.

### Functional Configuration of Medical Support Device

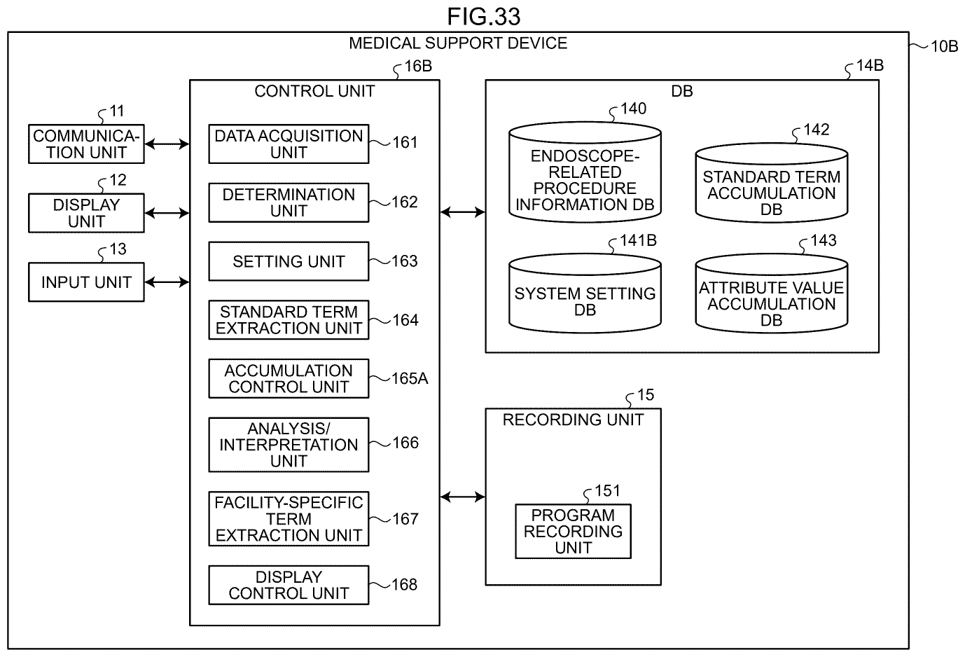

FIG. 33 is a block diagram illustrating a functional configuration of a medical support device according to the third embodiment of the present disclosure. The medical support device 10B illustrated in FIG. 33 includes a DB 14B and a control unit 16B, instead of the DB 14A and the control unit 16A of the medical support device 10A according to the second embodiment described above.

The DB 14B includes a system setting DB 141B, instead of the system setting DB 141 of the DB 14A according to the second embodiment described above.

The system setting DB 141B records, for every identifier of the system, system setting information in which a character division method, an attribute, and additional information extracted from the endoscope-related procedure information are associated.

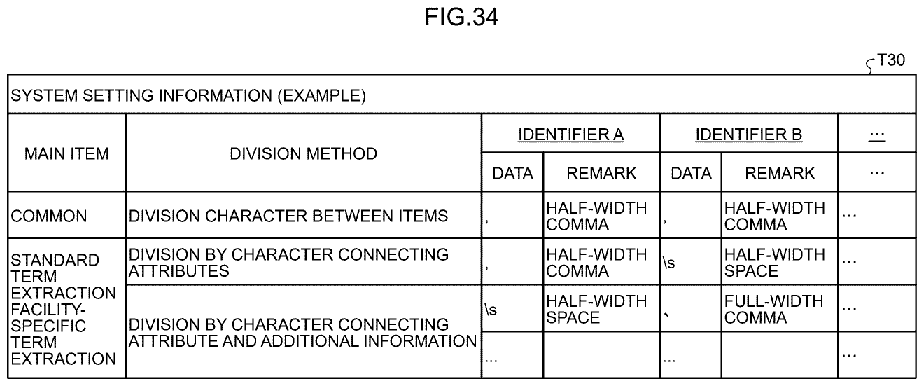

FIG. 34 is a diagram illustrating an example of system setting information recorded in the system setting DB 141B. The system setting information T30 illustrated in FIG. 34 stores, for each identifier of the system, as information for dividing and extracting, a connection character between items, a connection character between attributes, and a connection character between an attribute and additional information in the endoscope-related procedure information. Specifically, the system setting information T30 stores, for each of an identifier A and an identifier B, a division method “division character between items”, data “,”, and remark “half-width comma” in association with a main item “common”.

The control unit 16B corresponds to a processor according to the present disclosure. The control unit 16B is implemented by using a processor including hardware such as an FPGA, a GPU, or a CPU and a memory which is a temporary storage area used by the processor. In addition, the control unit 16B comprehensively controls each unit included in the medical support device 10B. The control unit 16B further includes a display control unit 168 in addition to the configuration of the control unit 16A according to the second embodiment described above.

The display control unit 168 displays, on the display unit 12, a warning that the setting unit 163 is not able to acquire, from the system setting DB 141B, the system setting information corresponding to the identifier which is for identifying the system and is included in a file.

### Processing of Medical Support Device

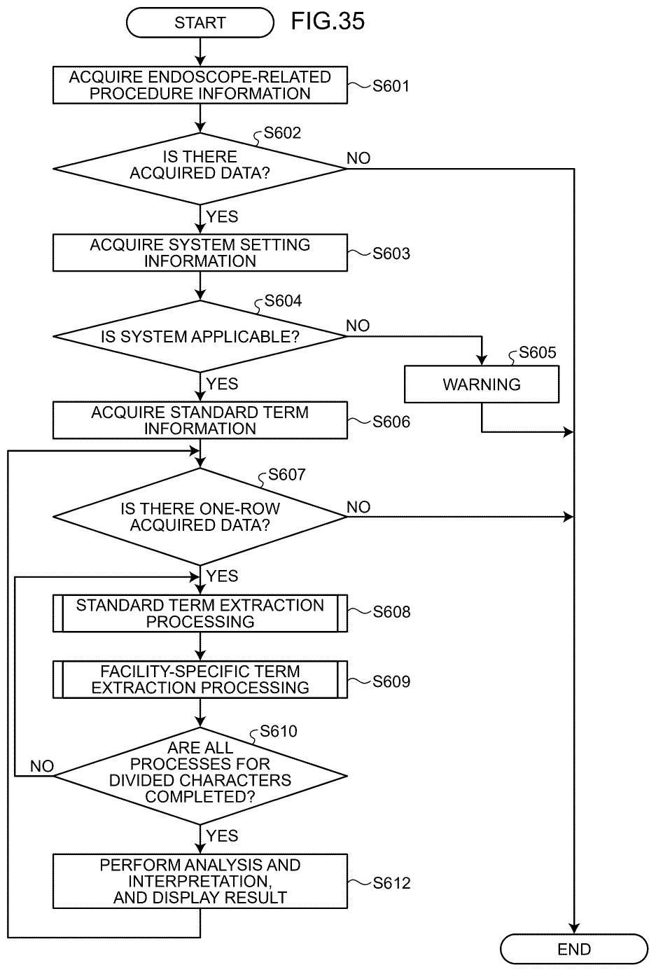

Next, processing executed by the medical support device 10B will be described. FIG. 35 is a flowchart illustrating an outline of processing executed by the medical support device 10B. In FIG. 35, step S602 and step S606 to step S611 are processes similar to step S302 and step S304 to step S309 in FIG. 21 described above, and step S601, step S603, step S604, and step S605 are different. Therefore, in the following description, step S601, step S603, step S604, and step S605 will be described respectively.

As illustrated in FIG. 35, the data acquisition unit 161 acquires the endoscope-related procedure information from the endoscope management device 20 via the communication unit 11 (step S601). In this case, the data acquisition unit 161 acquires, from the endoscope management device 20, a file including data in the JED form, which is the endoscope-related procedure information from the endoscope management device 20, and an identifier for identifying the system, via the communication unit 11.

In step S603, the setting unit 163 acquires, from the system setting DB 141B, the system setting information corresponding to the identifier which is for identifying the system and is included in the file.

Subsequently, the determination unit 162 determines whether or not the setting unit 163 is able to acquire, from the system setting DB 141B, the system setting information corresponding to the identifier which is for identifying the system and is included in the file (step S604). In a case where the determination unit 162 determines that the setting unit 163 is able to acquire, from the system setting DB 141B, the system setting information corresponding to the identifier which is for identifying the system and is included in the file (step S604: Yes), the medical support device 10B proceeds to step S606. On the other hand, in a case where the determination unit 162 determines that the setting unit 163 is not able to acquire, from the system setting DB 141B, the system setting information corresponding to the identifier which is for identifying the system and is included in the file (step S604: No), the medical support device 10B proceeds to step S605 to be described later.

In step S605, the display control unit 168 displays, on the display unit 12, a warning that the setting unit 163 is not able to acquire the system setting information corresponding to the identifier which is for identifying the system and is included in a file from the system setting DB 141B. Thereby, the user such as a doctor can recognize that the data in the JED form which is the endoscope-related procedure information from the endoscope management device 20 is incompatible data. After step S605, the medical support device 10B ends the processing.

According to the third embodiment described above, since the display control unit 168 displays, on the display unit 12, a warning indicating that the system setting information corresponding to the identifier which is for identifying the system and is included in the file is not acquired from the system setting DB 141B, the user such as a doctor can recognize that the data in the JED form which is the endoscope-related procedure information from the endoscope management device 20 is incompatible data.

In addition, according to the third embodiment, the setting unit 163 acquires the system setting information corresponding to the identifier which is for identifying the system and is included in the file, from the system setting DB 141B in which the system setting information is recorded. Thus, it is possible to perform system setting according to the system used in the facility.

### Fourth Embodiment

Next, a fourth embodiment of the present disclosure will be described. In the first to third embodiments described above, the standard term is sequentially updated, but in the fourth embodiment, the standard term is updated according to a certain use history (extraction history). Therefore, in the following description, a configuration of a medical support device according to the fourth embodiment will be described, and then processing executed by the medical support device according to the fourth embodiment will be described. Note that the same components as those of the medical support devices 10, 10A, and 10B according to the first to third embodiments described above are denoted by the same reference numerals, and a detailed description thereof will be omitted.

### Functional Configuration of Medical Support Device

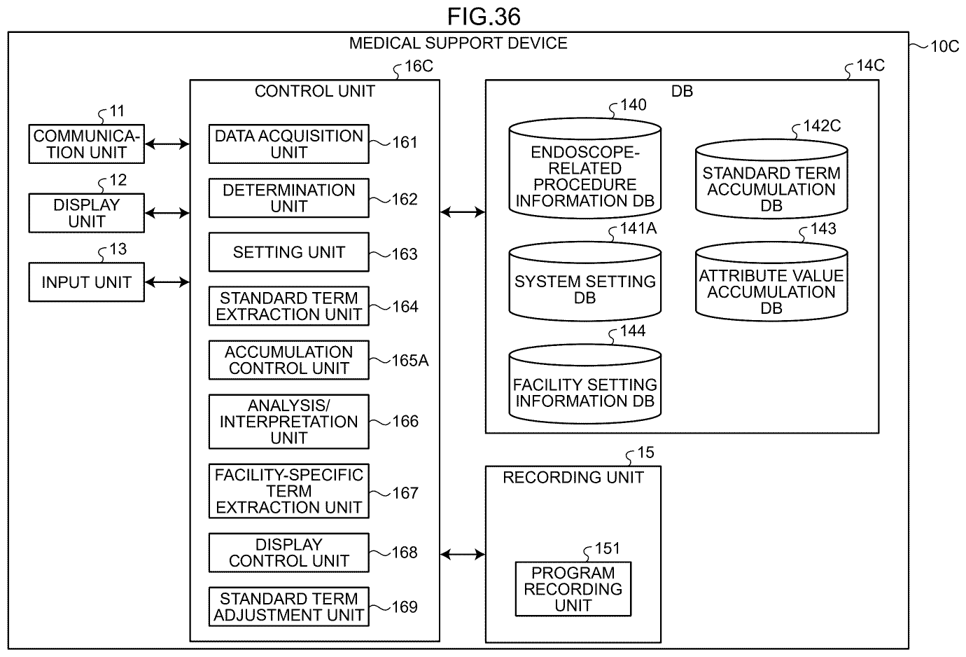

FIG. 36 is a block diagram illustrating a functional configuration of a medical support device according to the fourth embodiment of the present disclosure. The medical support device 10C illustrated in FIG. 36 includes a DB 14C and a control unit 16C, instead of the DB 14B and the control unit 16B of the medical support device 10B according to the third embodiment described above.

The DB 14C includes a standard term accumulation DB 142C instead of the standard term accumulation DB 142 of the DB 14B according to the third embodiment described above. Further, the DB 14C further includes a facility setting information DB 144 in addition to the configuration of the DB 14B according to the third embodiment described above.

In addition to the standard term (refer to the standard term T4 of FIG. 5 and the standard term T5 of FIG. 6) defined by the JED form described above, the standard term accumulation DB 142C further stores use information in which a category (standard) defined by the JED form or a category (specific) indicating a category extracted as a facility-specific term is associated with the number of uses of the facility-specific term.

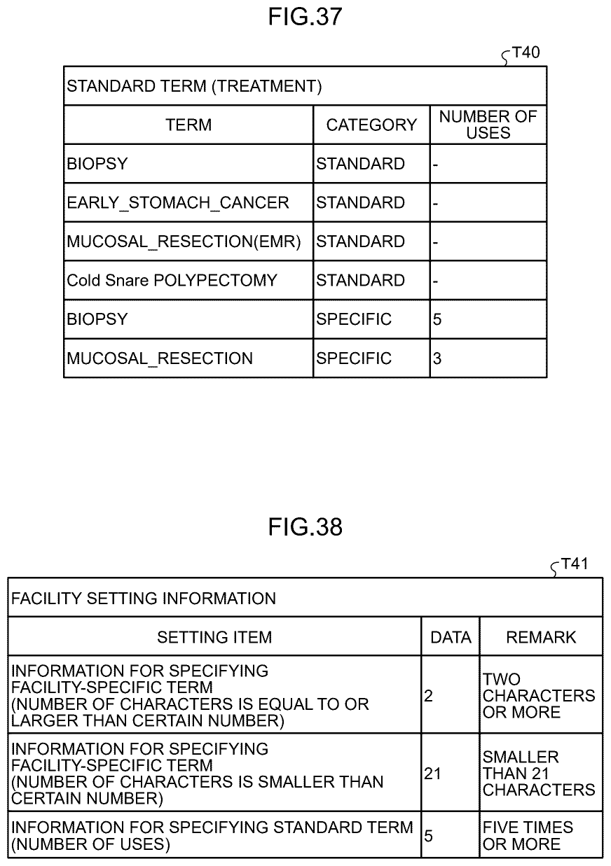

FIG. 37 is a diagram illustrating an example of standard term (treatment) use information. In the standard term (treatment) table T40 illustrated in FIG. 37, for the term of the standard term, a category (standard) defined by the JED form or a category (specific) indicating a category extracted as a facility-specific term is stored in association with the number of uses of the facility-specific term. For example, in the fifth line of the standard term T6, for the term “biopsy”, a category “specific” and the number of uses “5” are stored in association with each other.

The facility setting information DB 144 records facility setting information in which a setting item (item indicating whether the number of characters is equal to or larger than a certain number, or smaller than a certain number, or the like) for specifying a facility-specific term and information (a use history or the like) for specifying a standard term are associated for each facility.

FIG. 38 is a diagram illustrating an example of the facility setting information. In the facility setting information T41 illustrated in FIG. 38, data for specifying a standard term and a remark (the number of uses) are stored in association with a setting item (item indicating whether the number of characters is equal to or larger than a certain number, or smaller than a certain number, or the like) for specifying a facility-specific term (first condition). For example, the data “5” and the remark “five times or more” are stored in association with the setting item “information for specifying a standard term (number of uses)” in the third line of the facility setting information T41.

In addition, the facility setting information T41 includes the following second condition (2) and the following third condition (3) in addition to the first condition.

(Second Condition) The target character string is a front character string when the standard term extraction unit 164 divides the target character string by a character connecting attributes.

(Third Condition) The target character string does not include a character string that divides an attribute and additional information.

The control unit 16C corresponds to a processor according to the present disclosure. The control unit 16C is implemented by using a processor including hardware such as an FPGA, a GPU, or a CPU and a memory which is a temporary storage area used by the processor. In addition, the control unit 16C comprehensively controls each unit included in the medical support device 10C. The control unit 16C further includes a standard term adjustment unit 169 in the configuration of the control unit 16B according to the third embodiment.

The standard term adjustment unit 169 adjusts the standard terms by deleting the standard term from the standard term accumulation DB 142C in a case where the information (number of uses) for specifying the standard term is not satisfied according to the facility setting information which is set by the setting unit 163.

### Processing of Medical Support Device

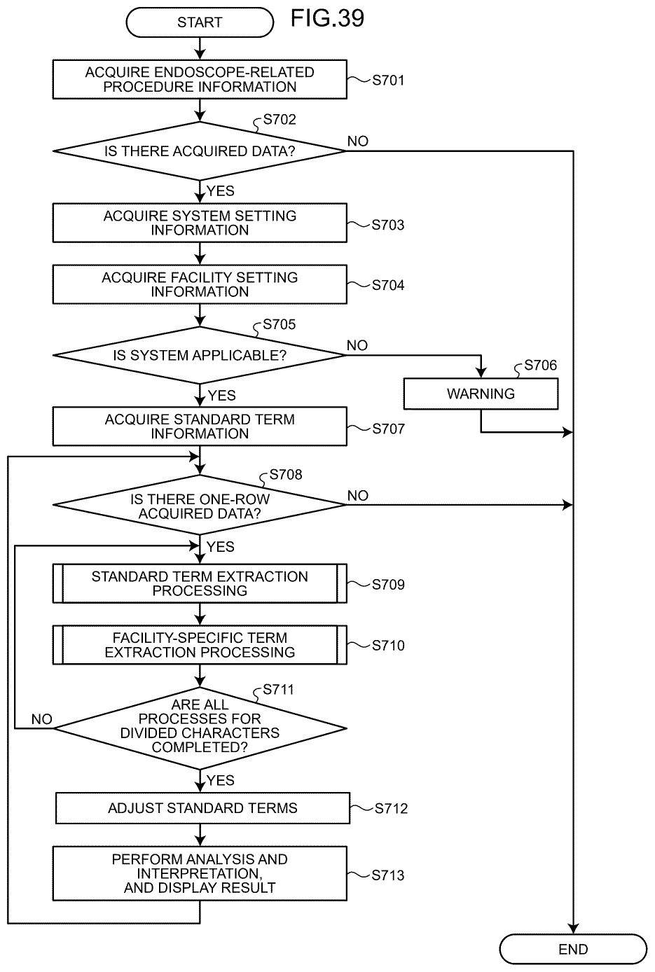

Next, processing executed by the medical support device 10C will be described. FIG. 39 is a flowchart illustrating an outline of processing executed by the medical support device 10C. In FIG. 39, step S701 to step S703, step S705 to step S708, step S711, and step S713 are processes similar to step S601 to step S603, step S604 to step S607, and step S610 and step S611 in FIG. 34 described above, and step S704, step S709, step S710, and step S712 are different. Therefore, in the following description, step S704, step S709, step S710, and step S712 will be described respectively.

In step S704, the setting unit 163 acquires the facility setting information from the facility setting information DB 144. After step S704, the medical support device 10C proceeds to step S705.

In step S709, the standard term extraction unit 164 executes standard term extraction processing of extracting a standard term from the one-row acquired data. This standard term extraction processing is similar to the standard term extraction processing of FIG. 9 described above, and is different only in counting up the number of uses (an extraction history or a use history).

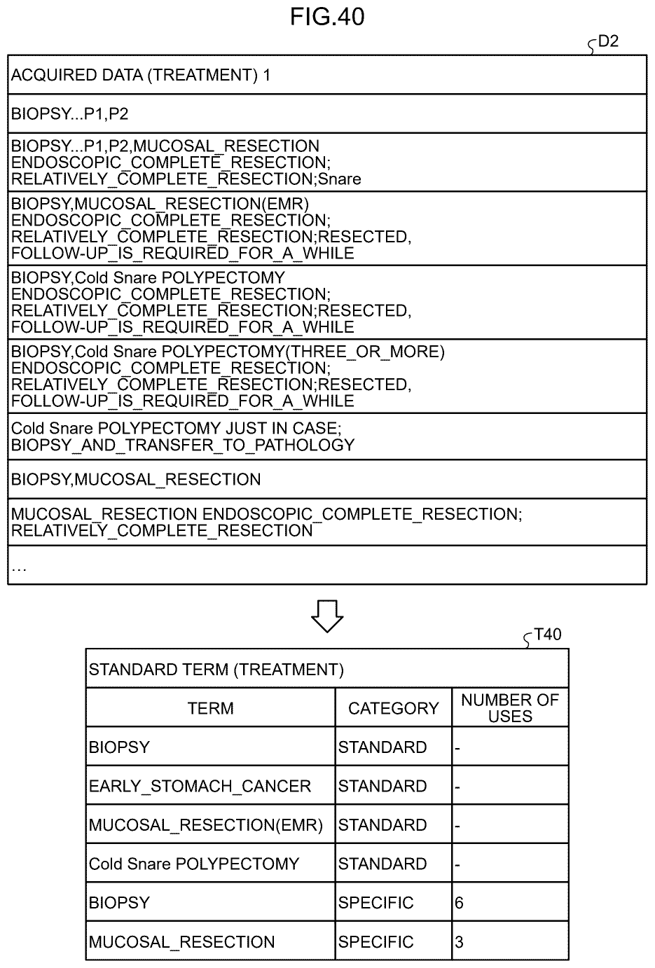

FIG. 40 is a diagram schematically illustrating a situation where the number of uses is counted up in a case where the standard term extraction unit 164 acquires an attribute extracted in the standard term extraction processing. As illustrated in FIG. 40, when the data acquisition unit 161 extracts an attribute value from the acquired data which is the endoscope-related procedure information and registers the attribute value in the attribute value accumulation DB 143, the standard term extraction unit 164 counts up the number of uses of the term, for example, the number of uses of “biopsy” in the standard term (treatment) table T40 from “5” to “6” and registers the number of uses of the term. In this case, according to the facility setting information which is set by the setting unit 163, since “Cold Snare polypectomy(three_or_more)” which is the acquired data (treatment) D2 does not correspond to information which is for specifying a facility-specific term and of which the number of characters is equal to or larger than two and smaller than 21, the standard term extraction unit 164 does not add the character string to the standard terms and ends the standard term extraction processing. After step S709, the medical support device 10C proceeds to step S710.

In step S710, the facility-specific term extraction unit 167 executes facility-specific term extraction processing of extracting a facility-specific term, on the acquired data (specific word) which is acquired by the data acquisition unit 161 and is divided by the standard term extraction unit 164.

### Facility-Specific Term Extraction Processing

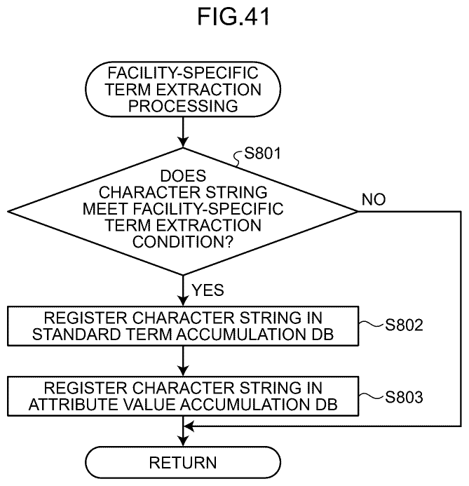

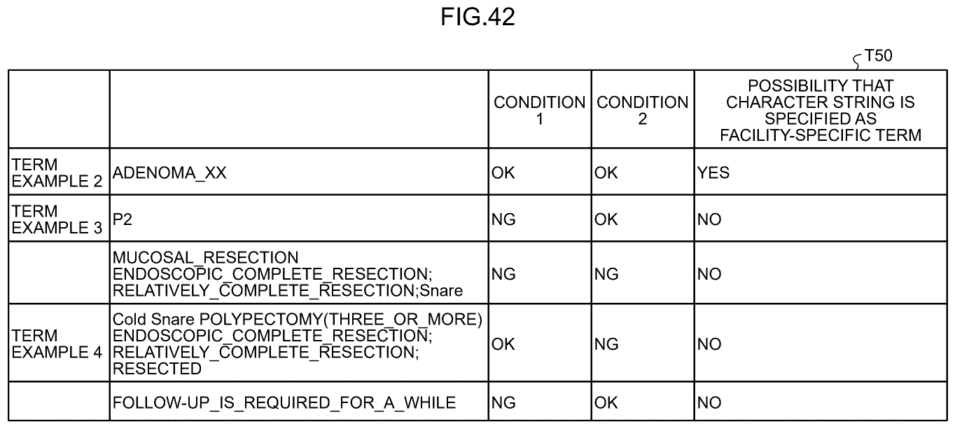

FIG. 41 is a sub-flowchart illustrating an outline of facility-specific term extraction processing in step S710 of FIG. 39. FIG. 42 is a diagram schematically illustrating an example of facility-specific term extraction processing by the facility-specific term extraction unit 167. Note that, in FIG. 41, step S802 and step S803 are similar to the processes of step S502 and step S503 in FIG. 27 described above, and only step S801 is different. Therefore, in the following description, step S801 will be described.

As illustrated in FIG. 41, the facility-specific term extraction unit 167 determines whether or not the specific word which is input from the standard term extraction unit 164 meets a facility-specific term extraction condition, according to the facility setting information (refer to the facility setting information T41 in FIG. 38) which is set by the setting unit 163 (step S801). Specifically, as illustrated in FIG. 28, the facility-specific term extraction unit 167 performs division on the basis of the half-width comma “,” and “ . . . ” of the system setting information T3 (refer to FIG. 4), divides the specific word “P2” and the specific word “mucosal_resection endoscopic_complete_resection;relatively_complete_resection;Snare”, which are divided and input by the standard term extraction unit 164, from a rear side, extracts and acquires “mucosal_resection” as a front character string, and determines whether or not “mucosal_resection” meets the facility-specific term extraction condition.

FIG. 42 is a diagram illustrating an example of an extraction result when the facility-specific term extraction unit 167 extracts a character string according to the facility setting condition. As illustrated in the extraction result table T50 in FIG. 42, the target character strings are “adenoma_X”, “P2”, “Cold Snare polypectomy(three_or_more) endoscopic_complete_resection;relatively_complete_resection;resected”, and “follow-up_is_required_for_a_while”, but these character strings are excluded from the targets by applying the second condition (2) and the third condition (3).

In addition, in the case of FIG. 26 described above, there is a possibility that the second and subsequent character strings divided by a character connecting attributes are either character strings which are divided in an unexpected form by characters of the free input portion or character strings which are divided in an unexpected form by a character connecting pieces of additional information (a connection character between attribute values “1” or attribute values “2” which are additional information, a connection character between an attribute value “1” and an attribute value “2”, and the like). Therefore, by setting only the front character string as a specific word, the facility-specific term extraction unit 167 can avoid registration of a character string which is divided in an unexpected form (a character string which is not expected as a facility-specific term).

Further, in the case of FIG. 26 described above, since the target character string “Cold Snare polypectomy (three or more), endoscopic complete resection, relatively complete resection, resected” includes “character that divides an attribute and additional information”, the facility-specific term extraction unit 167 does not specify the target character string as the facility-specific term. The reason is to cope with a case where the facility-specific term extraction unit 167 is not able to determine whether “character that divides an attribute and additional information” is included as the facility-specific term or “character that divides an attribute and additional information” is included as a data structure.

As a result, in the above case, the facility-specific term extraction unit 167 does not register the facility-specific term. Thereby, it is possible to avoid registration of a character string that is a specific word in an unexpected form.

Returning to FIG. 41, in step S801, in a case where the facility-specific term extraction unit 167 determines that the specific word which is input from the standard term extraction unit 164 meets the facility-specific term extraction condition (step S801: Yes), the medical support device 10C proceeds to step S502 to be described later. On the other hand, in a case where the facility-specific term extraction unit 167 determines that the specific word which is input from the standard term extraction unit 164 does not meet the facility-specific term extraction condition (step S801: No), the medical support device 10C returns to the main routine of FIG. 39, and proceeds to step S712.

Returning to FIG. 39, description of step S712 and subsequent steps will be continued.

In step S712, the standard term adjustment unit 169 adjusts the standard terms by deleting the standard term from the standard term accumulation DB according to the facility setting information which is set by the setting unit 163 in a case where the information (number of uses) for specifying the standard term is not satisfied.

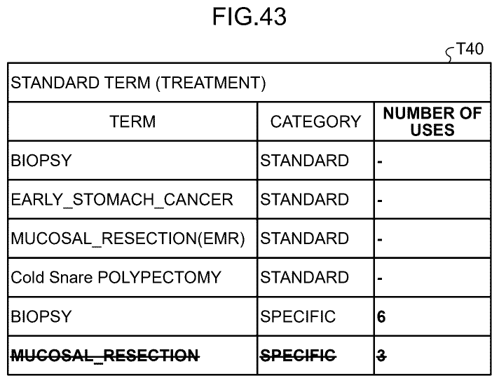

FIG. 43 is a diagram schematically illustrating adjustment of the standard term by the standard term adjustment unit 169. As illustrated in FIG. 43, the standard term adjustment unit 169 adjusts the standard terms by deleting, from the standard term (treatment) table T40, the term of which the number of uses is not equal to or larger than five, according to the facility setting information (refer to the facility setting information T41 in FIG. 25) which is set by the setting unit 163. For example, since the number of uses of “mucosal resection” in the standard term (treatment) table T40 is “3”, the standard term adjustment unit 169 adjusts the standard terms by deleting the standard term from the standard term (treatment) table T40. Thereby, it is possible to prevent an increase of terms with a low frequency of use, and to accurately extract terms. After step S712, the medical support device 10C proceeds to step S713.

According to the fourth embodiment described above, the standard term adjustment unit 169 adjusts the standard terms by deleting the standard term from the standard term accumulation DB according to the facility setting information which is set by the setting unit 163 in a case where the information (number of uses) for specifying the standard term is not satisfied. Thus, it is possible to avoid accumulation of unnecessary terms.

### Fifth Embodiment

Next, a fifth embodiment will be described. In the fourth embodiment described above, the number of uses of the standard term is counted, and in a case where the number of uses does not satisfy the information (number of uses) for specifying the standard term, the standard term is deleted from the standard term accumulation DB. On the other hand, in the fifth embodiment, a specific word extracted by the standard term extraction unit is temporarily accumulated, and it is determined whether or not the number of uses satisfies the information (number of uses) which is for specifying the standard term and is set by the facility setting unit every lapse of a specific period, and the standard term is registered in the standard term accumulation DB in a case where the number of uses satisfies the information (number of uses) for specifying the standard term. That is, in the fifth embodiment, the configuration of the medical support device and the standard term extraction processing executed by the medical support device are different, and processing of registering the standard term in the standard term accumulation DB is further included. Therefore, in the following description, after the configuration of the medical support device according to the fifth embodiment is described, the standard term extraction processing executed by the medical support device according to the fifth embodiment and processing of registering the standard term in the standard term accumulation DB will be described. Note that the same components as those of the medical support device 10, the medical support device 10A, the medical support device 10B, and the medical support device 10C according to the first to fourth embodiments are denoted by the same reference numerals, and a detailed description thereof will be omitted.

### Functional Configuration of Medical Support Device

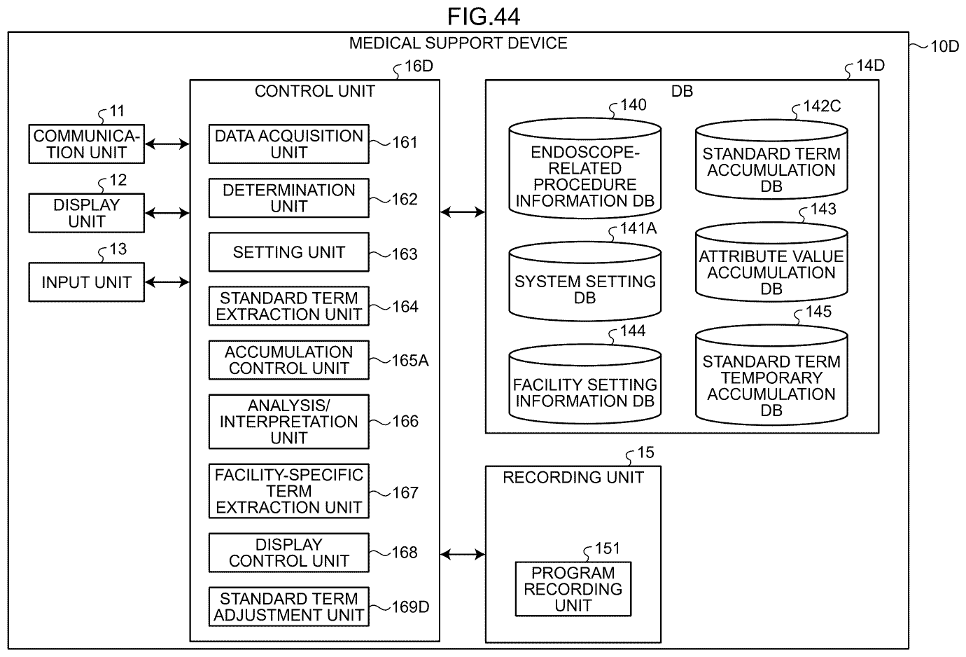

FIG. 44 is a block diagram illustrating a functional configuration of a medical support device according to the fifth embodiment of the present disclosure. The medical support device 10D illustrated in FIG. 44 includes a DB 14D and a control unit 16D, instead of the DB 14C and the control unit 16C of the medical support device 10C according to the fourth embodiment described above.

The DB 14D includes a standard term temporary accumulation DB 145 in addition to the configuration of the DB 14C according to the third embodiment.

The standard term temporary accumulation DB 145 temporarily accumulates a specific word extracted by the standard term extraction unit 164.

The control unit 16D corresponds to a processor according to the present disclosure. The control unit 16D is implemented by using a processor including hardware such as an FPGA, a GPU, or a CPU and a memory which is a temporary storage area used by the processor. In addition, the control unit 16D comprehensively controls each unit included in the medical support device 10C. The control unit 16D includes a standard term adjustment unit 169D instead of the standard term adjustment unit 169 of the control unit 16C according to the above-described fourth embodiment.

The standard term adjustment unit 169D determines whether or not the number of uses of the facility-specific term which is temporarily accumulated in the standard term temporary accumulation DB 145 is equal to or larger than a predetermined number of uses. In addition, the standard term adjustment unit 169D registers the facility-specific term, which is temporarily accumulated in the standard term temporary accumulation DB 145 and of which the number of uses is equal to or larger than a predetermined number of uses, in the standard term accumulation DB 142C, and does not register the facility-specific term of which the number of uses is not equal to or larger than a predetermined number of uses in the standard term accumulation DB 142C.

### Processing of Medical Support Device

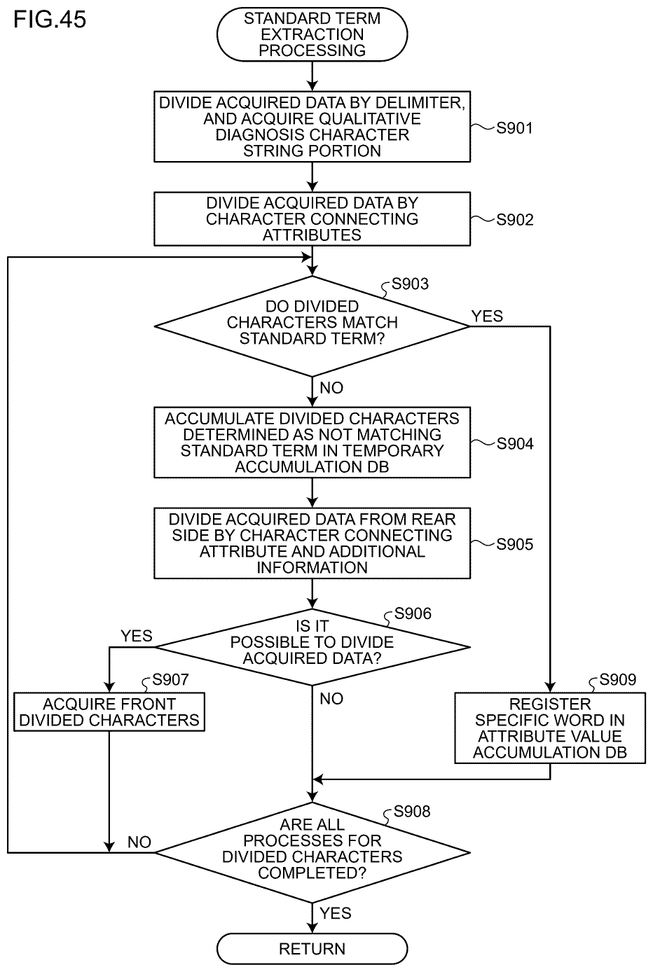

Next, processing executed by the medical support device 10D will be described. FIG. 45 is a flowchart illustrating an outline of standard term extraction processing executed by the medical support device 10D. In FIG. 45, step S901 and step S902 and step S905 to step S909 are processes similar to step S201 and step S202 and step S204 to step S208 in FIG. 9 described above, and only step S903, step S904, and step S908 are different. Therefore, in the following description, step S903, step S904, and step S908 will be described.

In step S903, the standard term extraction unit 164 determines whether or not the specific word matches the standard term which is accumulated in the standard term accumulation DB 142C. In a case where the standard term extraction unit 164 determines that the specific word matches the standard term which is accumulated in the standard term accumulation DB 142C (step S903: Yes), the medical support device 10D proceeds to step S909 to be described later. On the other hand, in a case where the standard term extraction unit 164 determines that the specific word does not match the standard term which is accumulated in the standard term accumulation DB 142C (step S903: No), the medical support device 10D proceeds to step S904 to be described later.

In step S904, the accumulation control unit 165A accumulates the specific word (facility-specific term) determined as not matching the standard term in step S903 described above in the standard term temporary accumulation DB 145. At this time, in a case where the same specific word has already been registered in the standard term temporary accumulation DB 145, the specific word may be accumulated so as to increase the number of uses. After step S904, the medical support device 10D proceeds to step S905.

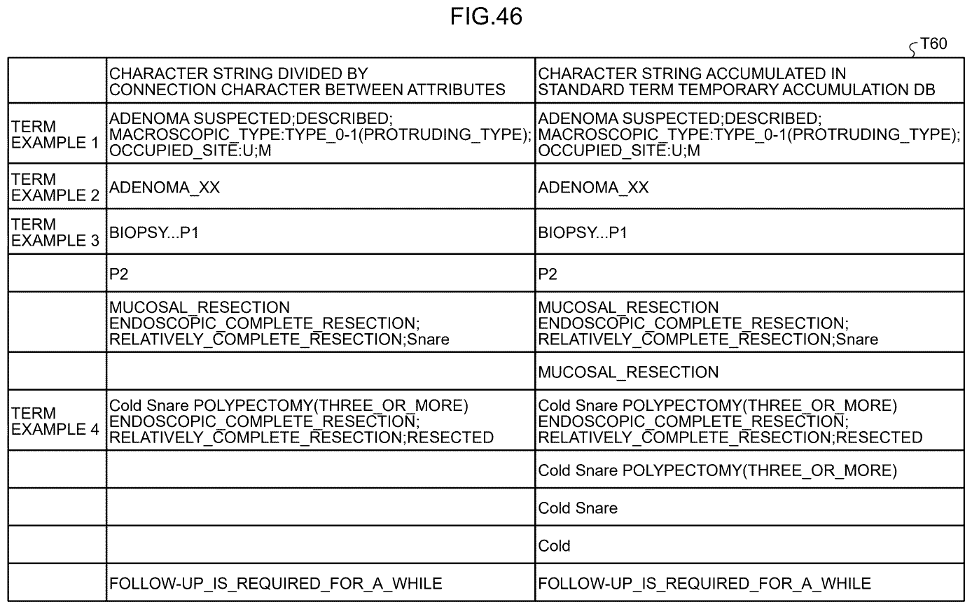

FIG. 46 is a diagram illustrating an example of a character string which is extracted by the standard term extraction unit 164 and is accumulated in the standard term temporary accumulation DB 145.

As illustrated in the extraction result table T60 of FIG. 46, a word corresponding to the standard term such as “Cold” or a part of the facility-specific term is also registered in the standard term temporary accumulation DB 145. However, in the processing by the standard term extraction unit 164, the endoscope-related procedure information is divided from a rear side by a character connecting an attribute and additional information, and a front character string among the divided characters is evaluated. Therefore, in a case where a long term including the word is registered, the word completely matches the long term. As a result, the standard term adjustment unit 169D to be described later sorts the standard terms by the number of uses. Thereby, it is possible to prevent an unnecessary term from being registered in the standard term accumulation DB 142C and to avoid generation of an unnecessary analysis result.

### Adjustment Processing by Standard Term Adjustment Unit

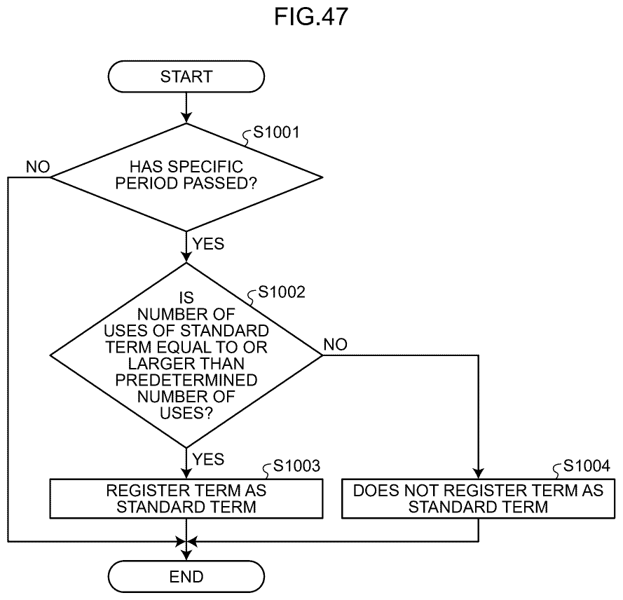

Next, adjustment processing executed by the standard term adjustment unit 169D will be described. FIG. 47 is a flowchart illustrating an outline of adjustment processing executed by the standard term adjustment unit 169D.

As illustrated in FIG. 47, the standard term adjustment unit 169D determines whether or not a specific period has passed for the facility-specific term which is temporarily accumulated in the standard term temporary accumulation DB 145 (step S1001). Here, the specific period can be appropriately set for each facility, and can be appropriately changed, for example, at night, one week, one month, one day, or the like. Of course, in addition to the period, the number of examinations, change of an operator, and the like may be taken into consideration. In a case where the standard term adjustment unit 169D determines that the specific period has passed for the facility-specific term which is temporarily accumulated in the standard term temporary accumulation DB 145 (step S1001: Yes), the medical support device 10D proceeds to step S1002 to be described later. On the other hand, in a case where the standard term adjustment unit 169D determines that the specific period has not passed for the facility-specific term which is temporarily accumulated in the standard term temporary accumulation DB 145 (step S1001: No), the medical support device 10D ends the processing.

In step S1002, the standard term adjustment unit 169D determines whether or not the number of uses of the facility-specific term which is temporarily accumulated in the standard term temporary accumulation DB 145 is equal to or larger than a predetermined number of uses. In a case where the standard term adjustment unit 169D determines that the number of uses of the facility-specific term which is temporarily accumulated in the standard term temporary accumulation DB 145 is equal to or larger than a predetermined number of uses (step S1002: Yes), the medical support device 10D proceeds to step S1003 to be described later. On the other hand, in a case where the standard term adjustment unit 169D determines that the number of uses of the facility-specific term which is temporarily accumulated in the standard term temporary accumulation DB 145 is not equal to or larger than a predetermined number of uses (step S1002: No), the medical support device 10D proceeds to step S1004 to be described later.

In step S1003, the standard term adjustment unit 169D registers, in the standard term accumulation DB 142C, the facility-specific term which is temporarily accumulated in the standard term temporary accumulation DB 145 and of which the number of uses is equal to or larger than a predetermined number of uses, and deletes the facility-specific term from the standard term temporary accumulation DB 145. After step S1003, the medical support device 10D ends the processing.

In step S1004, the standard term adjustment unit 169D does not register, in the standard term accumulation DB 142C, the facility-specific term which is temporarily accumulated in the standard term temporary accumulation DB 145 and of which the number of uses is not equal to or larger than a predetermined number of uses. In this case, the standard term extraction unit 164 may delete the facility-specific term which is temporarily accumulated in the standard term temporary accumulation DB 145 and of which the number of uses is not equal to or larger than a predetermined number of uses, or may add a log indicating that the facility-specific term is not adopted. After step S1004, the medical support device 10D ends the processing.

According to the fifth embodiment described above, the standard term adjustment unit 169D registers the facility-specific term, which is temporarily accumulated in the standard term temporary accumulation DB 145 and of which the number of uses is equal to or larger than a predetermined number of uses, in the standard term accumulation DB 142C, and does not register the facility-specific term of which the number of uses is not equal to or larger than a predetermined number of uses in the standard term accumulation DB 142C. Thus, it is possible to avoid accumulation of unnecessary terms and accumulation of attribute values of unnecessary terms (unnecessary analysis results).

In the fifth embodiment, in a case where the standard term is registered in step S1003, the accumulated previous endoscope-related procedure information may be processed again. By performing the retroactive processing, there is an effect that it is possible to perform analysis/interpretation on a period during which the term is temporarily accumulated and thus it is possible to update the analysis/interpretation result to be more appropriate. In addition, in order to efficiently perform the retroactive processing, it is useful to accumulate information indicating a hierarchy of data registered in the standard term temporary accumulation DB, a date, and the like, and to limit a range in which the processing is retroactively performed from when the term is registered in the standard term temporary accumulation DB until when the term is registered as a standard term.

### EMBODIMENTS OF DISCLOSURE

In the first to fifth embodiments, the analysis/interpretation unit 166 performs analysis/interpretation on the basis of the attribute value accumulation data which is accumulated in the attribute value accumulation DB 143, and displays, on the display unit 12, an analysis/interpretation result obtained by performing analysis/interpretation. On the other hand, the present disclosure is not limited thereto. The analysis/interpretation unit 166 may perform analysis/interpretation on various contents on the basis of information such as the endoscope-related procedure information acquired from the endoscope management device 20, and may display an analysis/interpretation result on the display unit 12.

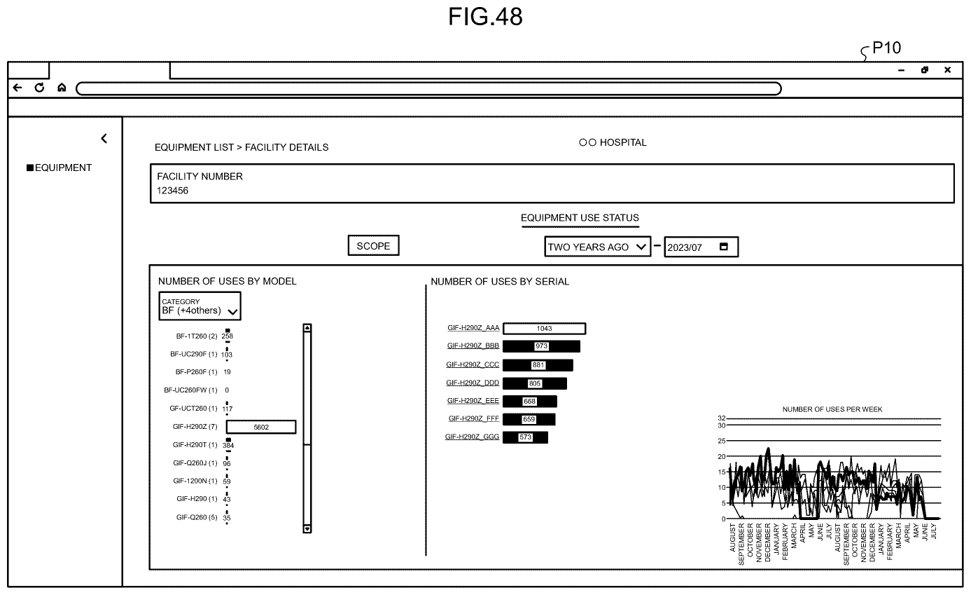

FIG. 48 is a diagram illustrating an example of an analysis/interpretation result analyzed/interpreted by the analysis/interpretation unit 166. As illustrated in FIG. 48, for example, the analysis/interpretation unit 166 may acquire a use history of the endoscope from the endoscope management device 20, analyze and interpret, for each model, the number of uses of the endoscope and the number of uses of the endoscope per week or per month on the basis of the use history, and display an analysis/interpretation result P10 on the display unit 12.

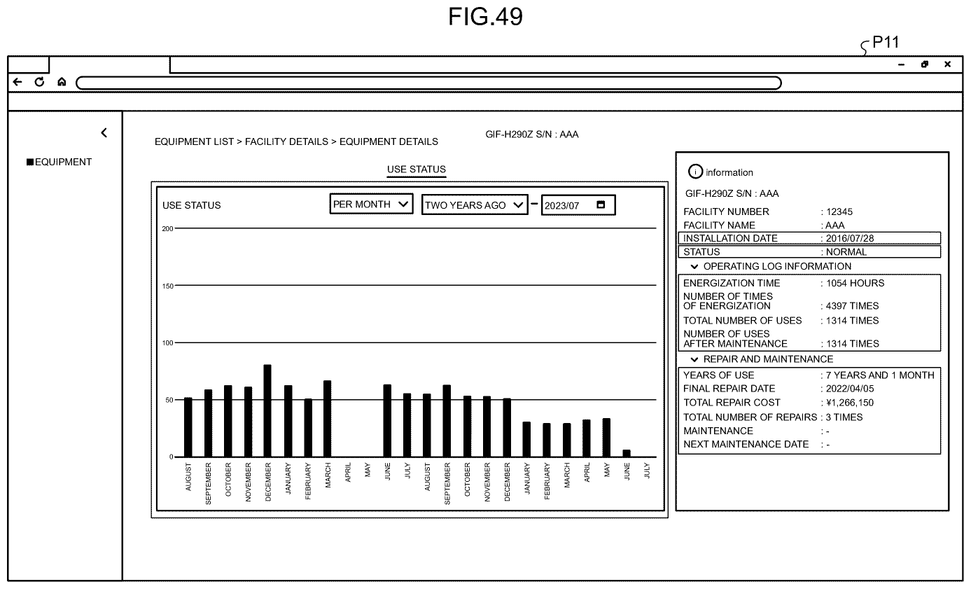

FIG. 49 is a diagram illustrating another example of an analysis/interpretation result analyzed/interpreted by the analysis/interpretation unit 166. As illustrated in FIG. 49, for example, the analysis/interpretation unit 166 may acquire a use history of the endoscope from the endoscope management device 20, analyze and interpret various types of information related to a status, an energization time, an energization frequency, the total frequency of use, the frequency of use after maintenance, and repair/maintenance on the basis of the use history, and display an analysis/interpretation result P11 on the display unit 12.

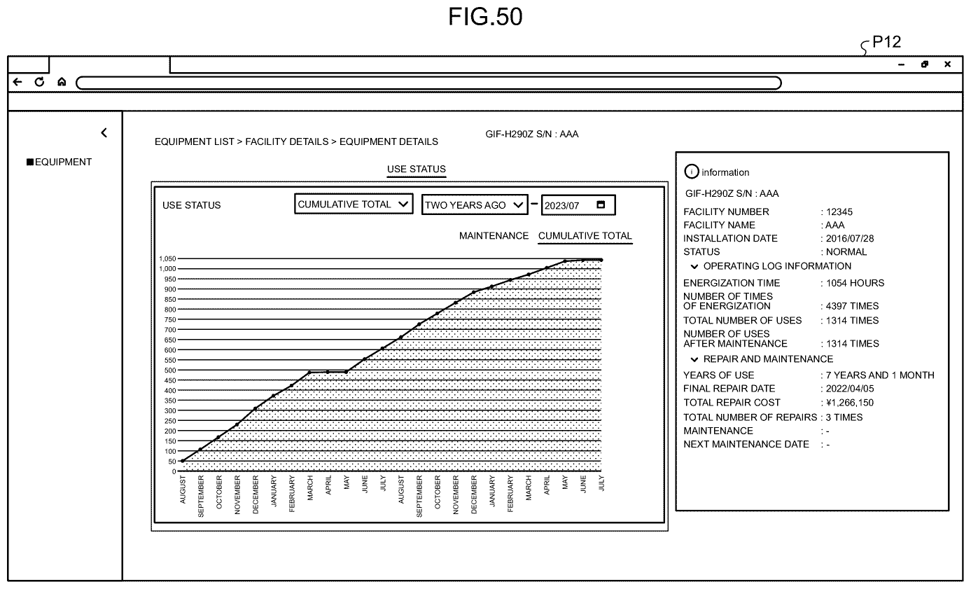

FIG. 50 is a diagram illustrating another example of an analysis/interpretation result analyzed/interpreted by the analysis/interpretation unit 166. As illustrated in FIG. 50, for example, the analysis/interpretation unit 166 may acquire a use history of the endoscope from the endoscope management device 20, analyze and interpret various types of information related to the cumulative number of uses of one endoscope on the basis of the use history, and display an analysis/interpretation result P12 on the display unit 12.

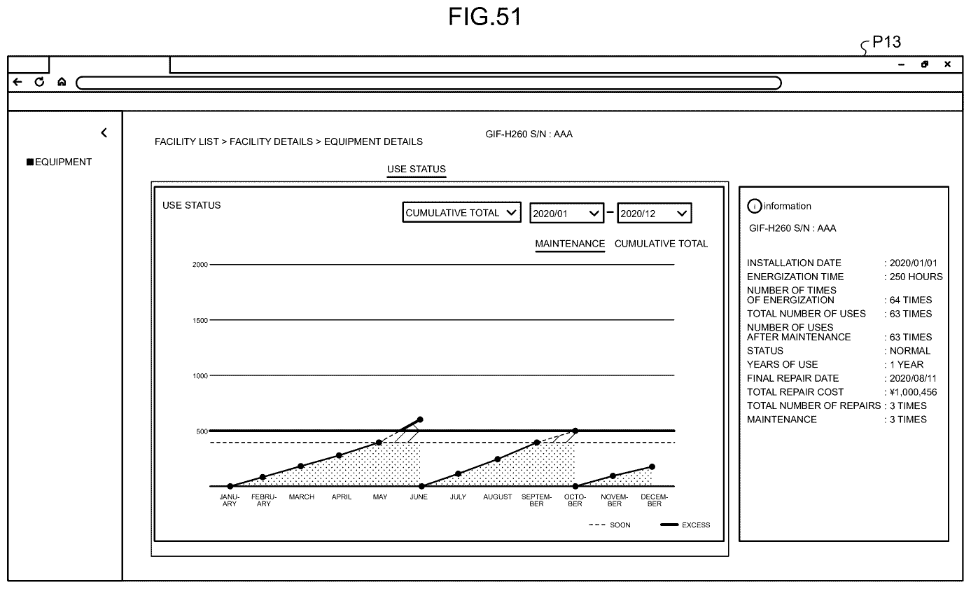

FIG. 51 is a diagram illustrating another example of an analysis/interpretation result analyzed/interpreted by the analysis/interpretation unit 166. As illustrated in FIG. 51, for example, the analysis/interpretation unit 166 may acquire a use history of an endoscope from the endoscope management device 20, analyze and interpret various types of information related to a maintenance situation of one endoscope on the basis of the use history, and display an analysis/interpretation result P13 indicating a period of periodic maintenance on the display unit 12.

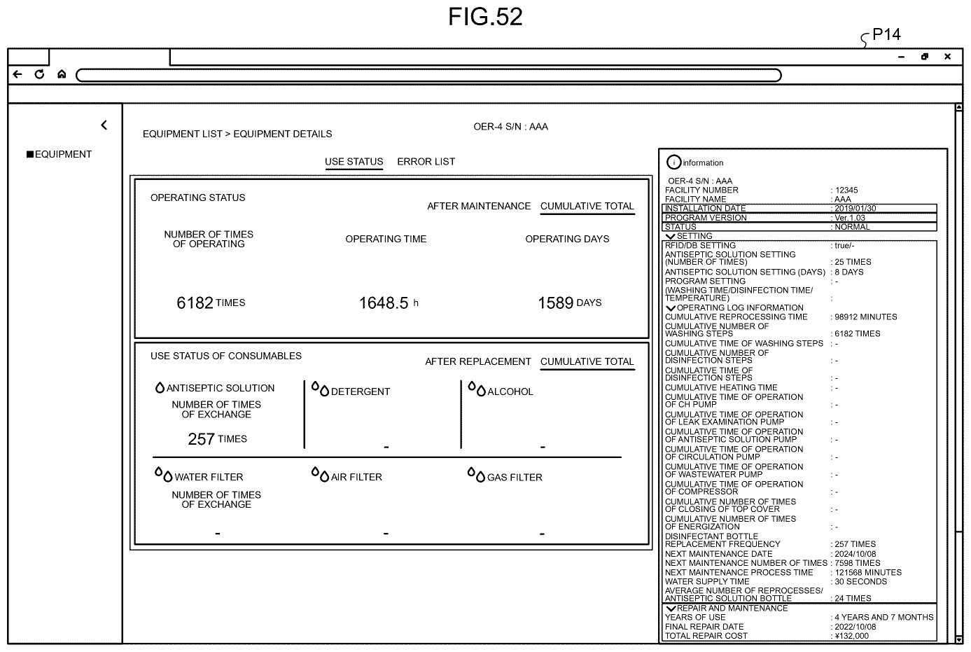

FIG. 52 is a diagram illustrating another example of an analysis/interpretation result analyzed/interpreted by the analysis/interpretation unit 166. As illustrated in FIG. 52, for example, the analysis/interpretation unit 166 may acquire a use history from an endoscope cleaning machine, analyze and interpret various types of information related to the endoscope cleaning machine on the basis of the use history, and display an analysis/interpretation result P14 obtained by visualizing an operation status of the endoscope cleaning machine on the display unit 12.

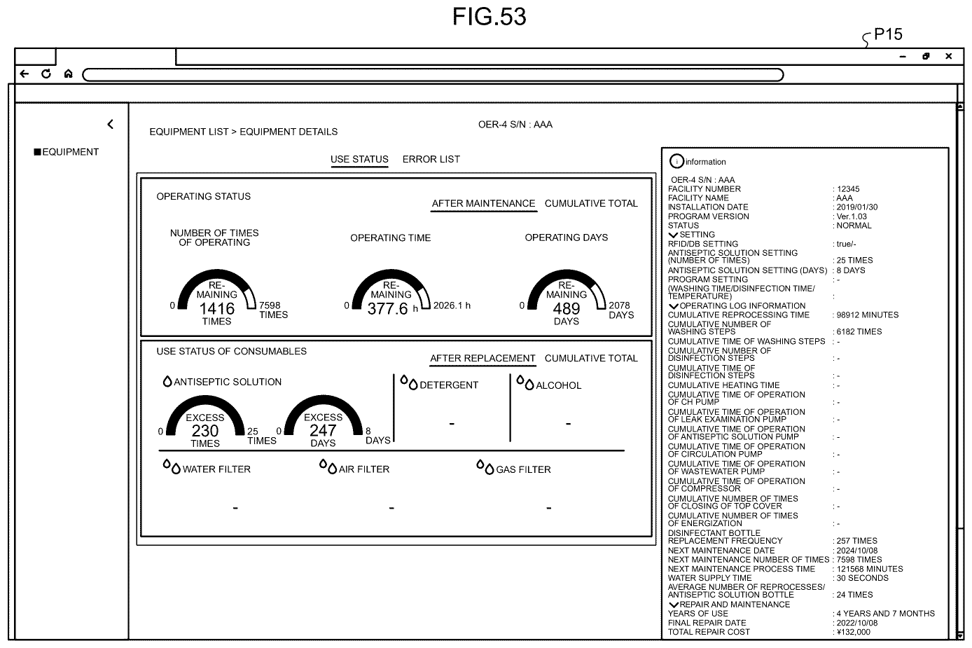

FIG. 53 is a diagram illustrating another example of an analysis/interpretation result analyzed/interpreted by the analysis/interpretation unit 166. As illustrated in FIG. 53, for example, the analysis/interpretation unit 166 may acquire a use history from an endoscope cleaning machine, analyze and interpret various types of information related to the endoscope cleaning machine on the basis of the use history, and display an analysis/interpretation result P15 obtained by visualizing an operation status of the endoscope cleaning machine on the display unit 12.

### OTHER EMBODIMENTS

Various inventions can be made by appropriately combining a plurality of components disclosed in the medical support system according to the embodiment of the present disclosure described above. For example, some components may be deleted from all the components described in the medical support system according to the embodiment of the present disclosure described above. Further, the components described in the medical support system according to the embodiment of the present disclosure described above may be appropriately combined.

Further, in the embodiment of the present disclosure, the medical support device includes the DB, but the present disclosure is not limited thereto. The medical support device and the DB on the server may be provided separately, and the determination results of the standard term extraction unit and the facility-specific term extraction unit may be transmitted to the DB on the server.

Further, in the embodiment of the present disclosure, each function of the control unit may be realized by a plurality of servers. For example, the function of the standard term extraction unit and the function of the accumulation control unit may be realized by separate servers.

In addition, in the embodiment of the present disclosure, the medical support device includes the function of the analysis/interpretation unit. However, for example, the function of the analysis/interpretation unit may be separately provided in a server, and the analysis/interpretation result may be acquired from the server by a service such as a subscription that periodically receives the analysis/interpretation result.

Further, in the medical support system according to the embodiment of the present disclosure, the above-described “unit” can be replaced with “means”, “circuit”, or the like. For example, the control unit can be replaced with control means or a control circuit.

In addition, the program to be executed by the medical support system according to the embodiment of the present disclosure is provided by being recorded in a computer-readable recording medium such as a CD-ROM, a flexible disk (FD), a CD-R, a digital versatile disk (DVD), a USB medium, or a flash memory, as file data in an installable form or an executable form.

Further, the program to be executed by the medical support system according to the embodiment of the present disclosure may be stored on a computer connected to a network such as the Internet, and may be provided by being downloaded via the network.

Note that, in the description of the flowcharts in the present specification, the order of the processing between steps is clearly indicated using expressions such as “first”, “thereafter”, and “subsequently”, but the order of the processing necessary for implementing the present invention is not uniquely determined by these expressions. That is, the order of the processing in the flowcharts described in the present specification can be changed within a range without inconsistency. In addition, the program is not limited to such a program including simple branch processing, and more determination items may be comprehensively determined and branched.

According to the present disclosure, an effect is achieved that it is possible to accurately extract terms used for analysis.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical support device comprising a processor comprising hardware, the processor being configured to:
perform division on endoscope-related procedure information with a division method according to content of the endoscope-related procedure information to extract a specific word;
compare the specific word with a standard term;
determine the specific word as an attribute value when the specific word matches the standard term;
determine and accumulate the specific word as a facility-specific term when the specific word does not match the standard term; and
update, as the standard term, the accumulated facility-specific term.

2. The medical support device according to claim 1, wherein
the processor is configured to:
count the number of uses of the specific word when the specific word is accumulated as the facility-specific term;
determine whether or not the number of uses of the specific word is equal to or larger than a predetermined number of times; and
perform control such that the specific word is not used as the facility-specific term when the number of uses of the specific word is smaller than the predetermined number of times.

3. The medical support device according to claim 1, wherein
the processor is configured to:
determine whether or not the number of uses of the specific word within a predetermined time is equal to or larger than a predetermined number of times; and
perform control such that the specific word is not used as the facility-specific term when the number of uses of the specific word is smaller than the predetermined number of times.

4. The medical support device according to claim 1, wherein
the division method includes a division method of performing division by a character connecting attributes, and a division method of performing division by a character connecting an attribute and additional information.

5. The medical support device according to claim 4, wherein
the processor is configured to:
perform the division on the endoscope-related procedure information from a rear side by the character connecting the attribute and the additional information;
sequentially acquire a front character string from among a plurality of character strings obtained from a result of the division; and
sequentially determine whether or not the front character string matches the standard term.

6. The medical support device according to claim 1, wherein
the processor is configured to:
determine whether or not the specific word that is a target character string satisfies a condition related to facility setting information indicating the number of characters for specifying a facility-specific term; and
determine the specific word as the facility-specific term when the condition is satisfied.

7. The medical support device according to claim 4, wherein
the processor is configured to:
determine whether or not the specific word that is a target character string satisfies at least one condition of a second condition or a third condition, the second condition indicating that the specific word is included in a front character string when the division is performed by at least the character connecting the attributes, the third condition indicating that the target character string does not include a character string which divides an attribute and additional information; and
determine the specific word as the facility-specific term when at least one condition of the second condition or the third condition is satisfied.

8. The medical support device according to claim 1, wherein
the endoscope-related procedure information further includes an identifier for identifying a system of the endoscope, and
the processor is configured to:
acquire system setting information of the division method corresponding to the identifier; and
extract the specific word based on the system setting information of the division method.

9. The medical support device according to claim 8, wherein
the processor is configured to:
determine whether or not the system setting information of the division method corresponding to the identifier is acquired; and
output a warning indicating that the system setting information of the division method corresponding to the identifier is not acquired when the system setting information of the division method corresponding to the identifier is not acquired.

10. The medical support device according to claim 1, wherein
the endoscope-related procedure information is information in a predetermined form, includes a plurality of characters, and is related to at least one of qualitative diagnosis or treatment by an endoscope.

11. A medical support method comprising:
performing division on endoscope-related procedure information with a division method according to content of the endoscope-related procedure information to extract a specific word;
comparing the specific word with a standard term;
determining the specific word as an attribute value when the specific word matches the standard term;
determining and accumulating the specific word as a facility-specific term when the specific word does not match the standard term; and
updating, as the standard term, the accumulated facility-specific term.

12. The medical support method according to claim 11, further comprising:
counting the number of uses of the specific word when the specific word is accumulated as the facility-specific term;
determining whether or not the number of uses of the specific word is equal to or larger than a predetermined number of times; and
performing control such that the specific word is not used as the facility-specific term when the number of uses of the specific word is smaller than the predetermined number of times.

13. The medical support method according to claim 11, further comprising:
determining whether or not the number of uses of the specific word within a predetermined time is equal to or larger than a predetermined number of times; and
performing control such that the specific word is not used as the facility-specific term when the number of uses of the specific word is smaller than the predetermined number of times.

14. The medical support method according to claim 11, wherein
the division method includes a division method of performing division by a character connecting attributes, and a division method of performing division by a character connecting an attribute and additional information.

15. The medical support method according to claim 14, further comprising:
performing the division on the endoscope-related procedure information from a rear side by the character connecting the attribute and the additional information;
sequentially acquiring a front character string from among a plurality of character strings obtained from a result of the division; and
sequentially determining whether or not the front character string matches the standard term.

16. The medical support method according to claim 11, further comprising:
determining whether or not the specific word that is a target character string satisfies at least one condition of a first condition, a second condition, or a third condition, the first condition being related to at least facility setting information indicating the number of characters for specifying a facility-specific term, the second condition indicating that the specific word is included in a front character string when the division is performed by a character connecting the attributes, the third condition indicating that the target character string does not include a character string which divides an attribute and additional information; and
determining the specific word as the facility-specific term when at least one condition of the first condition, the second condition, or the third condition is satisfied.

17. The medical support method according to claim 11, wherein
the endoscope-related procedure information is information in a predetermined form, includes a plurality of characters, and is related to at least one of qualitative diagnosis or treatment by an endoscope.

18. A non-transitory computer-readable recording medium with an executable program stored thereon, the program causing a processor of a medical support device to execute:
performing division on endoscope-related procedure information with a division method according to content of the endoscope-related procedure information to extract a specific word;
comparing the specific word with a standard term;
determining the specific word as an attribute value when the specific word matches the standard term;
determining and accumulating the specific word as a facility-specific term when the specific word does not match the standard term; and
updating, as the standard term, the accumulated facility-specific term.
